SEQUENCE LISTING (6 sequences)

SEQ ID NO: 1 - DNA, 1344 bp, Homo sapien
atgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccaggcgttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatga

SEQ ID NO: 2 - protein, 447 aa, Homo Sapien
MSLWLGAPVPDIPPDSAVELWKPGAQDASSQAQGGSSCILREEARMPHSAGGTAGVGLEAAEPTALLTRAEPPSEPTEIRPQKRKKGPAPKMLGNELCSVCGDKASGFHYNVLSCEGCKGFFRRSVIKGAHYICHSGGHCPMDTYMRRKCQECRLRKCRQAGMREECVLSEEQIRLKKLKRQEEEQAHATSLPPRRSSPPQILPQLSPEQLGMIEKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 3 - DNA, 1383 bp, Homo sapien
atgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacagcagcaggagtcacagtcacagtcgcagtcacctgtggggccgcagggcagcagcagctcagcctctgggcctggggcttcccctggtggatctgaggcaggcagccagggctccggggaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtga

SEQ ID NO: 4 - protein, 460 aa, Homo sapien
MSSPTTSSLDTPLPGNGPPQPGAPSSSPTVKEEGPEPWPGGPDPDVPGTDEASSACSTDWVIPDPEEEPERKRKKGPAPKMLGHELCRVCGDKASGFHYNVLSCEGCKGFFRRSVVRGGARRYACRGGGTCQMDAFMRRKCQQCRLRKCKEAGMREQCVLSEEQIRKKKIRKQQQESQSQSQSPVGPQGSSSSASGPGASPGGSEAGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 5 - DNA, 20 bp, Artificial Sequence
  LXR-alpha left primer
agtgtttgcacttcgcctgc

SEQ ID NO: 6 - DNA, 20 bp, Artificial Sequence
  LXR-alpha right primer
gtaagcttcagctgcgtggc

(19) United States
(12) Patent Application Publication
(10) Pub. No.: US 2005/0171084 A1
(43) Pub. Date: Aug. 4, 2005

(54) Methods of treatment with lxr modulators

(76) Inventors: William J Cairns, Harlow (GB); Elaine A. Irving, Harlow (GB); Andrew A. Parsons, Harlow (GB); Peter E. Soden, Harlow (GB); Jill C. Richardson, Harlow (GB); Stephen A. Burbidge, Harlow (GB); Mary Vinson, Harlow (GB); Mike A. Watson, Durham, NC (US); Karl D. Whitney, Durham, NC (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/509,197

(22) PCT Filed: Mar. 26, 2003
(86) PCT No.: PCT/US03/09225

Related U.S. Application Data
(60) Provisional application No. 60/368,424, filed on Mar. 27, 2002.

(51) Int. Cl. A61K 31/4439; A61K 31/4545; A61K 31/42; A61K 31/225

(52) U.S. Cl. 514/210.21; 514/346; 514/357; 514/394; 514/217.04; 514/318; 514/320; 514/379; 514/548

(57) ABSTRACT

Disclosed are methods of using compounds that modulate LXR.

### FIELD OF THE INVENTION

This invention relates to novel treatments and, in particular, to methods for the promotion of growth and/or repair of neurons in diseases or conditions characterised by neuron degeneration, injury or impaired plasticity.

### BACKGROUND OF THE INVENTION

The process of neurodegeneration is an important factor in many neurological diseases including acute disease such as stroke, traumatic brain injury and spinal cord injury as well as chronic disease including Alzheimer's disease, fronto-temporal dementias (tauopathies), peripheral neuropathy, Parkinson's disease, dementia with Lewy bodies, Huntington's disease, amyotrophic lateral sclerosis and multiple sclerosis. Agents offering neuroprotection, reduction of inflammatory response or enhancement of functional recovery may be useful in the treatment of these diseases. At present there are no treatments available which promote regeneration following neuronal damage due to such CNS diseases.

The mechanisms underlying functional recovery are currently unknown. Mechanisms thought to promote functional recovery include the sprouting of injured or non-injured axons, enhanced synaptic plasticity, differentiation of endogenous stem cells, activation of redundant pathways, changes in receptor distribution or excitability of neurons or glia (1,2).

Additionally, inflammation in the brain is increasingly seen as an important contributor to neurodegenerative disease mechanisms. Experimental and clinical damage to the brain leads to rapid upregulation of an array of pro-inflammatory mediators such as prostaglandin E2 (PGE2), tumour necrosis factor alpha (TNFα), nitric oxide (NO) and interleukin 6 (IL6). These factors are predominantly secreted by activated glia and exert many neurotoxic actions. Thus preventing or reducing inflammatory processes may also promote functional recovery (3).

Following the onset of stroke, some degree of spontaneous functional recovery is observed in many patients, suggesting that the brain has the ability to repair and/or remodel following injury. Agents that have the potential to enhance this recovery may therefore allow intervention to be made much later (potentially days) following the onset of cerebral ischaemia. Therapies that elicit axon sprouting following injury may therefore be valuable in restoring functional synaptic connections lost by the degenerating CNS in chronic and acute neurodegenerative diseases.

Finally, diseases where increased synaptic plasticity may also be beneficial are the psychiatric disorders including schizophrenia and depression. It has been reported that patients undergoing chronic treatment with effective anti-depressants display increased markers of synaptic plasticity. Compounds which enhance the ability of neurons to extend neurites and potentially increase neuroplasticity may therefore be effective in the prophylaxis and treatment of these disorders.

LXRα and LXRβ (collectively LXR) are nuclear hormone receptors that regulate the metabolism of several important lipids, including cholesterol (4). The nucleotide and amino acid sequences of LXRα are shown in FIGS. 3 and 4 (SEQ ID NOs:1 and 2), respectively. The nucleotide and amino acid sequences of LXRβ are shown in FIGS. 5 and 6 (SEQ ID NOs:3 and 4), respectively. The LXRs regulate the expression of target genes by binding to short stretches of DNA, termed LXRβ response elements (LXREs), as heterodimers with the retinoid X receptors (RXR)(5-8). LXREs have been identified in the regulatory regions of a number of genes involved in cholesterol homeostasis including CYP7A1 (9), which catalyses the first and rate-limiting step in bile acid biosynthesis, the cholesterol ester transport protein (10), the transcription factor SREBP-1C (11,12), apolipoprotein E (apoE)(13). LXREs have also been identified in the genes encoding the ATP binding cassette transporters (ABC) A1 and G1(14-18), which mediate the efflux of phospholipids and cholesterol from macrophages, intestinal enterocytes and other cell types.

Currently, patients with elevated levels of cholesterol are treated using the compounds that inhibit the body's endogenous cholesterol synthesis. As important components of the complex system that regulates cholesterol levels in the body the LXRs have also been proposed as targets for the prophylaxis and treatment of hypercholesteraemia (raised levels of plasma cholesterol) and its associated atherosclerotic diseases.

Schmidt, et al. (19) found that LXRβ activators 5-tetradecyloxy-2-furancarboxylic acid (TOFA) and 22(R)-hydroxycholesterol stimulated transcription from promoters under the control of AP-1 or NF-KB transcription factor binding sites and induced neuronal differentiation in rat pheochromocytoma cells.

It has now been found that LXR mRNA levels are elevated following transient middle cerebral artery occlusion (tMCAO) in the rat.

Administration of LXR agonists enhances neurite outgrowth in primary cultures of hippocampal and cortical neurons; limits the inflammatory response in microglial cells and upregulates the expression of LXR target genes in glial cells. LXR agonist administration also leads to increased cholesterol efflux from primary cell cultures of astrocytes and thus may promote synaptic plasticity.

The LXR target genes ABCA1, ApoE, ABCG1 and SREBP1c are known to be expressed in the CNS. In vivo the central administration of LXR agonists has been found to increase gene expression of some LXR target genes in the CNS.

### SUMMARY OF THE INVENTION

In one aspect, the present invention provides the use of an LXR agonist in the manufacture of medicaments for the treatment and/or prevention of diseases or conditions characterised by neuron degeneration; inflammation in the CNS, injury or impaired plasticity.

In another aspect, the present invention provides a method for treating a patient suffering from a disease selected from the group consisting of: stroke, Alzheimer's disease, fronto-temporal dementias, peripheral neuropathy, Parkinson's disease, dementia with Lewy bodies, Huntington's disease, amyotrophic lateral sclerosis, and multiple sclerosis, said method comprising the step of administering to said patient an effective amount of an LXR modulator in combination with a carrier.

In yet another aspect, the present invention provides a method for promoting cholesterol efflux in at least one astroglial cell, said method comprising the step of: contacting said at least one astroglial cell with a cholesterol-efflux-promoting effective amount of an LXR modulator in combination with a carrier.

### BRIEF DESCRIPTION OF THE DRAWINGS

FIG. 1 shows that LXR alpha mRNA levels were elevated in brains from tMCAO animals compared to sham-operated controls at 1 and 2 weeks post-surgery. The timepoints at which this elevation in mRNA levels is seen corresponds to the recovery period following MCAO in the rat in which a degree of spontaneous recovery is observed (20).

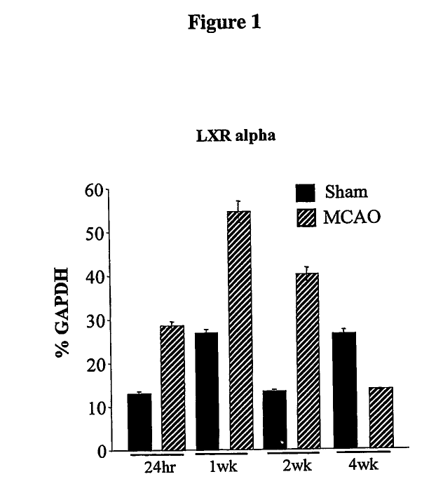

FIG. 2 shows that an LXR agonist (Example 1) can inhibit the secretion of pro-inflammatory mediators (IL-6, PGE2, TNF-α and NO) from LPS\INF-γ stimulated microglia cells.

FIG. 3 shows the nucleotide sequence of human LXRα (SEQ ID NO:1) from Genebank, accession NM_{—}005693.

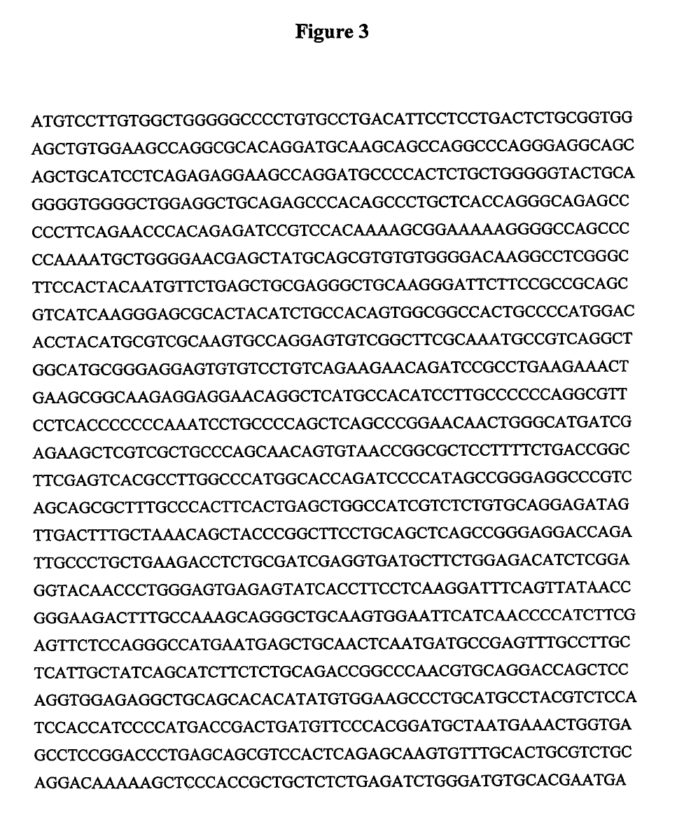

FIG. 4 shows the deduced amino acid sequence of human LXRα (SEQ ID NO:2) from Genebank accession NP_{—}005684.

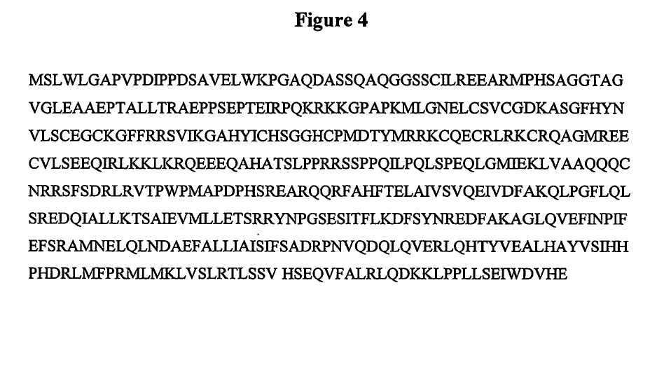

FIG. 5 shows the nucleotide sequence of human LXRβ (SEQ ID NO:3) from Genbank accession XM_{—}046419.

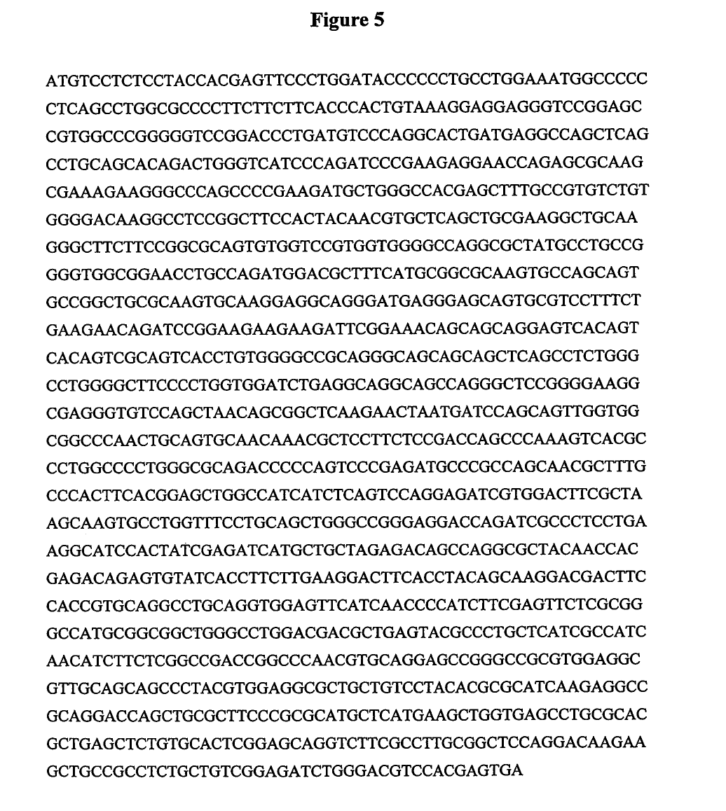

FIG. 6 shows the deduced amino acid sequence of human LXRβ (SEQ D NO:4) from Genebank accession XP_{—}046419.

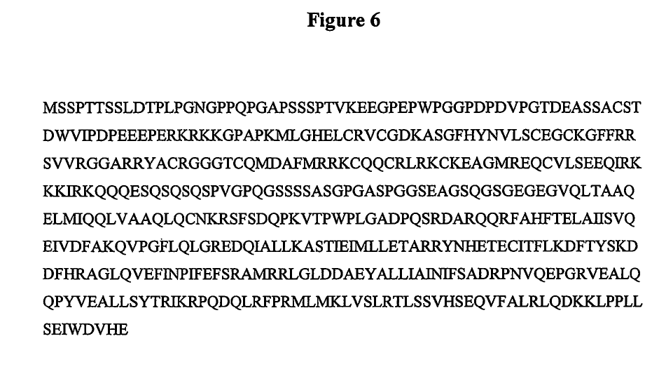

### DETAILED DESCRIPTION OF THE INVENTION

In a preferred aspect of the invention, the LXR agonists are selected from those disclosed in International Patent Applications WO 01/54759 (Tularik Inc. US), PCT/US01/27622 (SmithKline Beecham plc UK), WO 01/41704 (Merck & CO., INC) and WO97/28137 (Merck & CO., INC).

International Patent Application WO 00/54759 (Tularik Inc. US) discloses compounds of formula (1): 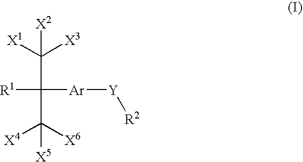
wherein:
Ar represents an aryl group; R¹ is —OH, —O—(C₁-C₇)alkyl, —OC(O)—(C₁-C₇)alkyl, —O—(C₁-C₇)heteroalkyl, —OC(O)— (C₁-C₇)heteroalkyl, —CO₂H, —NH₂, —NH(C₁-C₇)alkyl, —N((C₁-C₇)alkyl)₂ or —NH—S(O)₂—(C₁-C₅)alkyl;
R² is (C₁-C₇)alkyl, (C₁-C₇)heteroalkyl, aryl and aryl(C₁-C₇)alkyl;
X¹, X², X³, X⁴, X⁵ and X⁶ are each independently H, (C₁-C₅)alkyl, (C₁-C₅)hetroalkyl, F or Cl, with the proviso that no more than three of X¹ through X⁶ are H, (C₁-C₅)alkyl or (C₁-C₅)heteroalkyl; and
Y is —N(R¹²)S(O)ₘ—, —N(R¹²)S(O)ₘN(R¹³)—, —N(R¹²)C(O)—, —N(R¹²)C(O)N(R¹³)—, —N(R¹²)C(S)— or —N(R¹²)C(O)O—, wherein R12 and R13 are each independently hydrogen, (C₁-C₇)aryl, (C₁-C₇)heteroalkyl, aryl and aryl(C₁-C₇)alkyl, and optionally when Y is —N(R¹²)S(O)ₘ, or —N(R¹²)S(O)ₘN(R¹³)—, R¹² forms a five, six or seven-membered ring fused to Ar or to R² through covalent attachment to Ar or R², respectively. In the above Y groups, the subscript m is an integer of from 1 to 2,
   as being useful as agonists of LXR and their use in pharmaceutical formulations to reverse cholesterol transport and treat atherosclerotic cardiovascular diseases and related diseases.

With respect to the compounds of formula (I) the term “alkyl”, by itself or as part of another substituent, means, unless otherwise stated, a straight or branched chain, or cyclic hydrocarbon radical, or combination thereof, which may be fully saturated, mono- or polyunsaturated and can include di- and multi-radicals, having the number of carbons designated (i.e., C₁₋₁₀ means one to ten carbons). Examples of saturated hydrocarbon radicals include groups such as methyl, ethyl, n-propyl, isopropyl, n-butyl, t-butyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl”, unless otherwise noted, is also meant to include those derivatives of alkyl defined in more detail below as “cycloalkyl” and “alkylene”. The term “alkylene” by itself or as part of another substituent means a divalent radical derived from alkane, as exemplified by —CH₂CH₂CH₂CH₂—. Typically, an alkyl group will have from 1 to 24 carbon atoms, with those having 10 or fewer carbon atoms being preferred. A “lower alkyl” or “lower alkylene” is a shorter chain alkyl or alkylene group, generally having eight or fewer carbon atoms, preferably four or fewer carbon atoms.

The term “alkoxy”, employed alone or in combination with other terms means, unless otherwise stated, an alkyl group, as defined above, connected to the remainder of the molecule via an oxygen atom, such as, for example, methoxy, ethoxy, 1-propoxy, 2-propoxy, and the higher homologs and isomers.

The term “heteroalkyl”, by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si, S, and wherein the nitrogen and sulfur atoms may optionally be oxidized and the nitrogen heteroatom may optionally be quarternized. The heteroatom(s) O, N and S may be placed at any position of the heteroalkyl group except for the position at which the alkyl group is attached to the remainder of the molecule. Examples include —CH₂—CH₂—O—CH₃, —CH₂—CH₂—NH—CH₃, —CH₂—CH₂—N(CH₃), —CH₂—S—CH₂—CH₃, —CH₂—CH₂—S(O)—CH₃, —CH₂—CH₂—S(O)₂—CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Also included in the term “heteroalkyl” are those radicals described in more detail below as “heteroalkylene” and “heterocycloalkyl.” The term “heteroalkylene by itself or as part of another substituent means a divalent radical derived from heteroalkyl, as exemplified by —CH₂—CH₂—S—CH₂—CH₂— and —CH₂—S—CH₂—CH₂—NH—CH₂—. For heteroalkylene groups, heteroatoms can also occupy either or both of the chain termini. Still further, for alkylene and heteroalkylene linking groups, as well as all other linking groups described herein, no specific orientation of the linking group is implied.

The terms “cycloalkyl” and “heterocycloalkyl”, by themselves or in combination with other terms, represent, unless otherwise stated, cyclic versions of “alkyl” and “heteroalky” respectively. The terms “cycloalkyl” and “heterocycloalkyl” are also meant to include bicyclic, tricyclic and polycyclic versions thereof. Additionally, for heterocycloalkyl, a heteroatom may occupy the position at which the heterocyclyl is attached to the remainder of the molecule. Examples of cycloalkyl include cyclopentyl, cyclohexyl, 1-cyclohexyl, 3-cyclohexyl, cyclopentyl, bicyclo[2.2.1]heptyl, bicyclo[2.2.2]octyl, adamantyl, and the like. Example of heterocycloalkyl include 1-(1,2,5,6-tetrahydropyridyl), 1-piperidinyl, 2-piperidinyl, 3-piperidinyl, 4-morpholinyl, 3-morpholinyl, 1,4-diazabicyclo[2.2.2]oct-2-yl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen” by themselves or as part of another substituent, mean, unless otherwise stated, a fluorine, chlorine, bromine or iodine atom. Additionally, terms such as “fluoroalkyl”, are meant to include monofluoroalkyl and polyfluoroalkyl.

The term “aryl”, employed alone or in combination with other terms (e.g., aryloxy, arylthioxy, arylalkyl) means, unless otherwise stated, an aromatic substituent which can be a single ring or multiple rings (up to three rings) which are fused together or linked covalently. The rings may each contain from zero to four heteroatoms selected from N, O and S, wherein the nitrogen and sulfur atoms are optionally oxidized, and the nitrogen atom(s) are optionally quaternized. The aryl groups that contain heteroatoms may be referred to as “heteroaryl” and can be attached to the remainder of the molecule through a carbon atom or a heteroatom. Non-limiting examples of aryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidinyl, 4-pyrimidinyl, 5-benzothiazolyl, purinyl, 2-benzimidazolyl, 5-indolyl, 1-isoquinolinyl, 5-isoquinolinyl, 2-quinoxalinyl, 5-quinoxalinyl, 3-quinolinyl, and 6-quinolinyl. Substituents for each of the above noted aryl ring systems are selected form the group of acceptable substituents described below.

The terms “arylalkyl” and “arylheteroalkyl” are meant to include those radicals in which an aryl group is attached to an aryl group (e.g., benzyl, phenethyl, pyridylmethyl and the like) or a heteroalkyl group (e.g. phenoxymethyl, 2-pyridyloxymethyl, 1-napthyloxy-3-propyl, and the like). The arylaklyl and arylheteroalkyl groups will typically contain from 1 to 3 aryl moieties attached to the alkyl or heteroalkyl portion by a covalent bond or by fusing the ring to, for example, a cycloalkyl or heterocycloalkyl group. For arylheteroalkyl groups, a heteroatom can occupy the position at which the group is attached to the remainder of the molecule. For example, the term “arylheteroalkyl” is meant to include benzyloxy, 2-phenylethoxy, phenethylamine, and the like.

Each of the above terms (e.g., “alkyl”, “heteroalkyl”, “aryl” etc) is meant to include both substituted and unsubstituted forms of the indicated radical. Preferable substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and hetercycloalkenyl) can be a variety of groups selected from: —OR, ═O, ═NR′, N—OR′, NR′R″, —SR′, -halogen, —SiR′RR′″, —OC(O)R′, —CO_{2R}′; —CONR′R″, OC(O)NR′R″, —NR″C(O)R′, —NR″C(O)NR′R″, —NR″C(O)₂R′, NHC(NH₂)═NH, —NR′C(NH₂)═NH, —NH—, C(NH₂)═NR′, S(O)R′, —S(O)₂R″, —S(O)₂NR′R″, —CN and —NO₂ in a number ranging from zero to (2N+1), where N is the total number of carbon atoms in such a radical. Preferably, substituted alkyl groups will have from one to six independently selected substituents, more preferably from one to four independently selected substituents, most preferably from one to three independently selected substituents. In the substituents listed above, R′, R″ and R′″ each independently refer to hydrogen, unsubstituted (C₁₋₈)alkyl and heteroalkyl, unsubstituted aryl, aryl substituted with 1-3 halogens, unsubstituted alkyl, alkoxy or thioalkoxy groups or aryl-(C₁₋₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl.

Similarly, substituents for the aryl groups are varied and selected from: -halogen, —OR′, —OC(O)R′, —NR′R″, —SR′, —R′, —CN, —NO₂, —CO₂R′, —CONR′R″, —OC(O)NR′R″, —NR″C(O)R′, —NR″C(O)₂R′, —NR″C(O)NR′R′″, —NH—C(NH₂)═NH, —NR′C(NH₂)═NH, —NH—C(NH₂)═NR′, —SOR′, —S(O)₂R′, —S(O)₂NR′R″, —N₃, —CH(Ph)₂, perfluor(C₁₋₄)alkoxy, and perfluoro(C₁₋₄)alkyl, in a number ranging from zero to the total number of open valences on the aromatic ring system; and where R′ and R″ are independently selected from hydrogen, (C₁₋₈)alkyl and heteroalkyl, unsubstituted aryl, (unsubstituted aryl)-(C₁₋₄)alkyl, and (unsubstituted aryl)oxy-(C₁₋₄)alkyl. Preferably, substituted aryl groups will have from one to four independently selected substituents, more preferably from one to three independently selected substituents, most preferably from one to two independently selected substituents.

Two of the substituents on adjacent atoms of the aryl ring may optionally be replaced with a substituent of the formula -T-C(O)—(CH₂)_{q}-U-, wherein T and U are independently —NH—, —O—, CH₂ or a single bond, and q is an integer of from 0 to 2. Alternatively, two of the substituents on adjacent atoms of the aryl ring may optionally be replaced with a substituent of formula -A-(CH₂)r-B—, wherein A and B are independently —CH₂—, —O—, —NH—, S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl ring may optionally be replaced with a substituent of the formula —(CH₂)_{S}—X—(CH₂)ₜ—, where s and t are integers of from 0 to 3, and X is —O—, —NR′—, —S—, —S(O)—, —S(O)₂—, or —S(O)₂NR′—. The substituent R′ in —NR′— and S(O)₂NR′— selected from hydrogen or unsubstituted (C₁₋₆)alkyl.

The term “heteroatom” is meant to include oxygen (O), nitrogen (N), sulfur (S) and silicon (Si).

The term, “LXR modulator,” as used herein, means a small molecule that modulates the biological activities of LXRα and/or LXRβ. More specifically, such an LXR modulator either enhances or inhibits the biological activities of LXR. If such a modulator partially or completely enhances the biological activities of LXR, it is a partial or complete LXR agonist, respectively. Conversely, if such a modulator either partially or completely inhibits the biological activities of LXR, it is a partial or complete LXR antagonist, respectively.

Example 1 of WO 00/54759 (Tularik Inc. US) has the following structure:

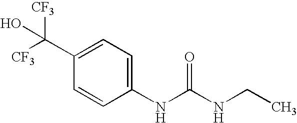

Compounds of formula (I) can be prepared using readily available starting materials or known intermediates. WO 00/54759 describes a number of possible synthetic routes for the production of such compounds, such as those depicted in scheme 1.

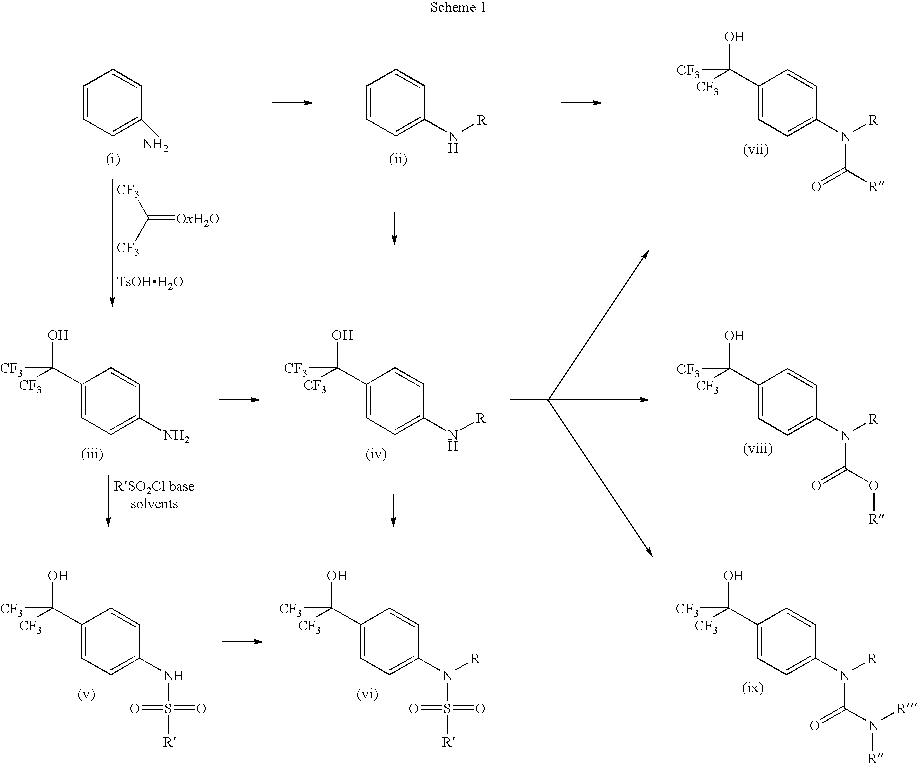

As shown in Scheme 1, aniline (I) (as representative of substituted anilines and other arylamines) can be alkylated, acylated or arylated (general addition of R group) to form (ii), or the aromatic ring can be derivatized with, for example, hexafluoroacetone to form (iii). Treatment of (iii) with an appropriate alkylating group, acylating group or arylating group provides (iv), which can be sulfonylated with, for example, an appropriate sulfonyl halide to form (vi). Alternatively, the aniline derivative can be sufonylated to form (v), which can then be alkylated or acylated to form compounds of formula (vi).

Other compounds of formula (I) can be formed by treating the substituted aniline (iv) (or iii), with reagents suitable for the formation of amides (vii), carbamates (viii) and ureas (ix). Various reagents are useful in the above scheme and can be found in, for example March, Advanced Organic Chemistry 4th ed. John Wiley & Sons, New York N.Y. (1992).

International Patent Application PCT/US01/27622 (SmithKline Beecham plc) discloses compounds of formula (II): 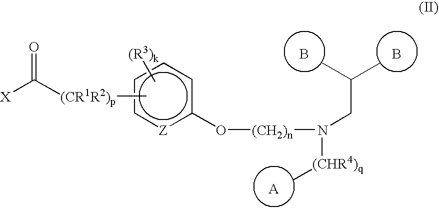
wherein:
X is OH or NH₂;
p is 0-6;
each R¹ and R² are the same or different and are each independently selected from the group consisting of H, C₁₋₈alkyl, C₁₋₈alkoxy and C₁₋₈thioalkyl;
Z is CH or N;
when Z is CH, k is 0-4;
when Z is N, k is 0-3;
each R³ is the same or different and is independently selected from the group consisting of halo, —OH, C₁₋₈alkyl, C₂₋₈alkenyl, C₁₋₈alkoxy, C₂₋₈alkenyloxy, —S(O)ₐR⁶, —NR⁷R⁸, —COR⁶, COOR⁶, R¹⁰COOR⁶, OR¹⁰COOR⁶, CONR⁷R⁸, —OC(O)R⁹, —R¹⁰NR⁷R⁸, —OR¹⁰NR⁷R⁸, 5-6 membered heterocycle, nitro, and cyano;
   a is 0, 1 or 2;
   R⁶ is selected from the group consisting of H, C₁₋₈alkyl, C₁₋₈alkoxy and C₂₋₈alkenyl;
   each R⁷ and R⁸ are the same or different and are each independently selected from the group consisting of H, C₁₋₈alkyl, C₂₋₈alkenyl, C₃₋₈alkynyl;
   R⁹ is selected from the group consisting of H, C₁₋₈alkyl and —NR⁷R⁸;
   R¹⁰ is C₁₋₈alkyl;
n is 2-8;
q is 0 or 1;
R⁴ is selected from the group consisting of H, C₁₋₈alkyl, C₁₋₈alkenyl, and alkenyloxy;
Ring A is selected from the group consisting of C₈cycloalkyl, aryl, 4-8 membered heterocycle, and 5-6 membered heteroaryl;
each ring B is the same or different and is independently selected from the group consisting of C₃₋₈cycloalkyl and aryl,
as being useful as agonists of LXR and their use in pharmaceutical formulations to reverse cholesterol transport and treat atherosclerotic cardiovascular diseases and related diseases.

With respect to compounds of formula (II) the term “alkyl” refers to aliphatic straight or branched saturated hydrocarbon chains containing the specified number of carbon atoms. Examples of “alkyl” groups as used herein include but are not limited to methyl, ethyl, n-propyl, isopropyl, n-butyl, isobutyl, t-butyl, pentyl, hexyl, octyl and the like. The term “alkyl” also refers to substituted alkyl wherein the substituents are selected from the group consisting of halo, —OR⁷ and —SR⁷, where R⁷ is H or C₁₋₈alkyl. This definition of “alkyl” is also applicable to terms such as “thioalkyl” which incorporate the “alkyl” term. Thus, a “thioalkyl” as used herein refers to the group S—Ra where Ra is “alkyl” as defined.

The term “halo” refers to any halogen atom ie., fluorine, chlorine, bromine or iodine.

The term “alkenyl” refers to an aliphatic straight or branched unsaturated hydrocarbon chain containing at least one and up to three carbon-carbon double bonds. Examples of “alkenyl” groups as used herein include, but are not limited to, ethenyl and propenyl. The term “alkenyl” also refers to substituted alkenyl wherein the substituents are selected from the group consisting of halo, —OR⁷ and —SR⁷, where R⁷ is H or C₁₋₈alkyl.

The term “alkoxy” refers to a group O—Ra where Ra is “alkyl” as defined above.

The term “alkenyloxy” refers to a group O—Rb where Rb is “alkenyl” as defined above.

The term “cycloalkyl” refers to a non-aromatic carbocyclic ring having the specified number of carbon atoms and up to three carbon-carbon double bonds. “Cycloalkyl” includes by way of example cyclopropyl, cyclobutyl, cyclopentyl, cyclohexyl, cycloheptyl, cyclooctyl, cyclobutenyl, cyclopentenyl, cyclohexenyl and bicyclic cycloalkyl groups such as bicycloheptane and bicyclo(2.2.1)heptene. The term “cycloalkyl” also refers to substituted cycloalkyl wherein the ring bears one or more substituents selected from the group consisting of halo, —OH, C₁₋₈alkyl, C₂₋₈alkenyl, C₁₋₈alkoxy, C₂₋₈alkenyloxy, S(O)ₐR⁶, —NR⁷R⁸, —COR⁶, —COOR⁶, —R¹⁰COOR⁶, —OR¹⁰COOR⁶, —CONR⁷R⁸, —OC(O)R⁹, —R¹⁰NR⁷R⁸, —OR¹⁰NR⁷R⁸, nitro, and cyano, wherein a is 0, 1 or 2; R⁶ is selected from the group consisting of H, C₁₋₈alkyl, C₁₋₈alkoxy and C₂₋₈alkenyl; each R⁷ and R⁸ is the same or different and is independently selected from the group consisting of H, C₁₋₈alkyl, C₂₋₈alkenyl and C₃₋₈alkynyl; R⁹ is selected from the group consisting of H, C₁₋₈alkyl and —NR⁷R⁸; and R¹⁰ is C₁₋₈alkyl. As will be appreciated by those skilled in the art, the number of possible substituents on the cycloalkyl ring will depend upon the size of ring. In one preferred embodiment, the cycloalkyl is a cyclohexyl which may be substituted as described above.

The term “aryl” refers to aromatic groups selected from the group consisting of phenyl, 1-naphthyl and 2-naphthyl. The term “aryl” also refers to substituted aryl wherein the phenyl or naphthyl ring bears one or more substituents selected from the group consisting of halo, —OH, C₁₋₈alkyl, C₂₋₈alkenyl, C₁₋₈alkoxy, C₂₋₈alkenyloxy, S(O)ₐR⁶, —NR⁷R⁸, —COR⁶, —COOR⁶, —R¹⁰COOR⁶, —OR¹⁰COOR⁶, —CONR⁷R⁸, —OC(O)R⁹, —R¹⁰NR⁷R⁸, —OR¹⁰NR⁷R⁸, nitro, and cyano, wherein a is 0, 1 or 2; R⁶ is selected from the group consisting of H, C₁₋₈alkyl, C₁₋₈alkoxy and C₂₋₈alkenyl; each R⁷ and R⁸ is the same or different and is independently selected from the group consisting of H, C₁₋₈alkyl, C₂₋₈alkenyl and C₃₋₈alkynyl; R⁹ is selected from the group consisting of H, C₁₋₈alkyl and —NR⁷R⁸; and R¹⁰ is C₁₋₈alkyl. As will be appreciated by those skilled in the art, the number of possible substituents on the aryl ring will depend upon the size of ring. For example, when the aryl ring is phenyl, the aryl ring may have up to 5 substituents selected from the foregoing list. One skilled in the art will readily be able to determine the maximum number of possible substituents for a 1-naphthyl or 2-naphthyl ring. A preferred aryl ring according to formula (II) is phenyl, which may be substituted as described above.

The term “heterocycle” refers to a monocyclic saturated or unsaturated non-aromatic carbocyclic rings and fused bicyclic non-aromatic carbocyclic rings, having the specified number of members in the ring and containing 1, 2 or 3 heteroatoms selected from N, O and S. Examples of particular heterocyclic groups include but are not limited to tetrahydrofuran, dihydropyran, tetrahydropyran, pyran, oxetane, thietane, 1,4-dioxane, 1,3-dioxane, 1,3-dioxalane, piperidine, piperazine, tetrahydropyrimidine, pyrrolidine, morpholine, thiomorpholine, thiazolidine, oxazolidine, tetrahydrothiopyran, tetrahydrothiophene, and the like. The term “heterocycle” also refers to substituted heterocycles wherein the heterocyclic ring bears one or more substituents selected from the group consisting of halo, —OH, C₁₋₈alkyl, C₂₋₈alkenyl, C₁₋₈alkoxy, C₂₋₈alkenyloxy, S(O)ₐR⁶, —NR⁷R⁸, —COR⁶, —COOR⁶, —R¹⁰COOR⁶, —OR¹⁰COOR⁶, —CONR⁷R⁸, —OC(O)R⁹, —R¹⁰NR⁷R⁸, —OR¹⁰NR⁷R⁸, nitro, and cyano, wherein a is 0, 1 or 2; R⁶ is selected from the group consisting of H, C₁₋₈alkyl, C₁₋₈alkoxy and C₂₋₈alkenyl; each R⁷ and R⁸ is the same or different and is independently selected from the group consisting of H, C₁₋₈alkyl, C₂₋₈alkenyl and C₃₋₈alkynyl; and R⁹ is selected from the group consisting of H, C₁₋₈alkyl and —NR⁷R⁸; and R¹⁰ is C₁₋₈alkyl. As will be appreciated by those skilled in the art, the number of possible substituents on the heterocyclic ring will depend upon the size of ring. There are no restrictions on the positions of the optional substituents in the heterocycles. Thus, the term encompasses rings having a substituent attached to the ring through a heteroatom. One skilled in the art will readily be able to determine the maximum number and locations of possible substituents for any given heterocycle. A preferred heterocycle according to the invention is piperidine, which may be substituted as described above.

The term “heteroaryl” refers to aromatic monocyclic heterocyclic rings and aromatic fused bicyclic rings having the specified number of members in the ring, having at least one aromatic ring and containing 1, 2 or 3 heteroatoms selected from N, O and S. Examples of particular heteroaryl groups include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, and indazole. The term “heteroaryl” also refers to substituted heteroaryls wherein the heteroaryl ring bears one or more substituents selected from the group consisting of halo, —OH, C₁₋₈alkyl, C₂₋₈alkenyl, C₁₋₈alkoxy, C₂₋₈alkenyloxy, S(O)ₐR⁶, —NR⁷R⁸, —COR⁶, —COOR⁶, —R¹⁰COOR⁶, —OR¹⁰COOR⁶, —CONR⁷R⁸, —OC(O)R⁹, —R¹⁰NR⁷R⁸, —OR¹⁰NR⁷R⁸, nitro, and cyano, wherein a is 0, 1 or 2; R⁶ is selected from the group consisting of H, C₁₋₈alkyl, C₁₋₈alkoxy and C₂₋₈alkenyl; each R⁷ and R⁸ is the same or different and is independently selected from the group consisting of H, C₁₋₈alkyl, C₂₋₈alkenyl and C₃₋₈alkynyl; and R⁹ is selected from the group consisting of H, C₁₋₈alkyl and —NR⁷R⁸; and R¹⁰ is C₁₋₈alkyl. As will be appreciated by those skilled in the art, the number of possible substituents on the heteroaryl ring will depend upon the size of ring. There are no restrictions on the positions of the optional substituents in heteroaryls. Thus, the term encompasses rings having a substituent attached to the ring through a heteroatom. One skilled in the art will readily be able to determine the maximum number and locations of possible substituents for any given heteroaryl. A preferred heteroaryl according to the invention is pyridine, which may be substituted as described above.

The term “protecting group” refers to suitable protecting groups useful for the synthesis of compounds of formula (I) wherein X is OH. Suitable protecting groups are known to those skilled in the art and are described in Protecting Groups in Organic Synthesis, 3^{rd} Edition, Greene, T. W.; Wuts, P. G. M. Eds.; John Wiley & Sons: NY, 1999. Examples of preferred protecting groups include but are not limited to methyl, ethyl, benzyl, substituted benzyl, and tert-butyl. In one embodiment the protecting group is methyl.

Example 16 of PCT/US01/27622 (Smith Kline Beecham plc) has the following structure:

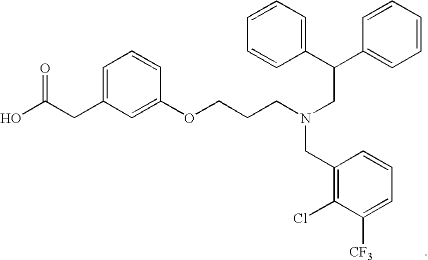

Compounds of formula II can be made according to any suitable method of organic chemistry. One method given in the specification is a solid phase synthesis process as depicted in Scheme 2. 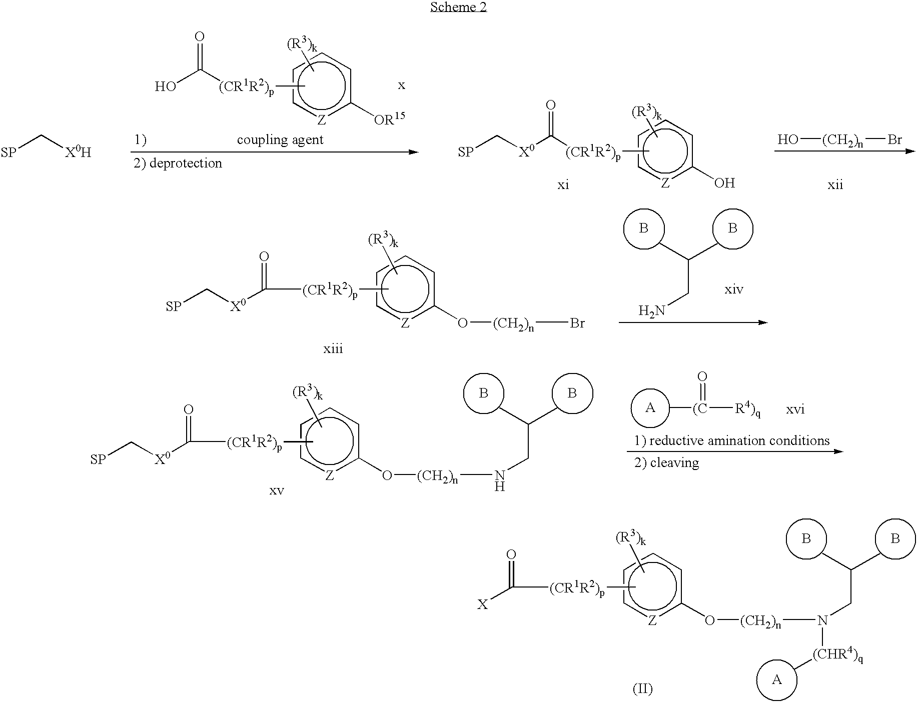
wherein X⁰ is —O— or —NH—, SP is solid phase, R¹⁵ is H or a protecting group, and all other variables are as defined above in connection with the description of compounds of formula (II).

In general, the reaction proceeds by a) reacting a solid phase-bound amine (where X in the compound of formula (II) is NH₂) or alcohol (where X in the compound of formula (II) is OH) with a compound of formula (x) and a coupling agent to produce a solid phase-bound compound of formula (xi); b) in the embodiment wherein R¹⁵ is a protecting group, deprotecting the solid phase bound compound to prepare the compound of formula (xi); c) alkylating the solid phase-bound compound of formula (xi) with an alcohol of formula (xii) to produce a solid phase-bound compound of formula (xiii); d) reacting the solid-phase-bound compound of formula (xiii) with a compound of formula (xiv) to produce the solid-phase bound compound of formula (xv); and e) reacting the solid phase-bound compound of formula (xv) with a compound of formula (xvi) under reductive amination conditions to produce the solid phase-bound compound of formula (II). The process may optionally further comprise the step of cleaving the solid phase-bound compound of formula (II) from the solid phase using conventional techniques such as treatment with mild acid.

Compounds of formula (II) are commercially available or can be prepared using conventional techniques such as those described in European Patent No. 303,742.

Compounds of formula (II) are commercially available or can be prepared using conventional techniques such as those described in European Patent No. 303,742.

Compounds of formula (III) are described in U.S. Provisional Application Nos. 09/368,427, 60/368,425 and 60/368,426, each filed Mar. 27, 2002: 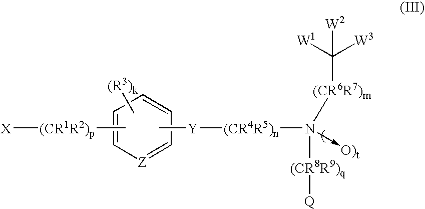
wherein:
X is selected from C₁-C₈ alkyl, halo, —OR¹⁰, —NR¹⁴R¹⁵, nitro, cyano, —COOR¹⁰, —COR¹³, —OCOR¹³, —CONR¹⁴R¹⁵, —N(R¹⁷)COR¹³, —N(R¹⁷)CONR¹⁴R¹⁵, —N(R¹⁷)COOR¹³, —SO₃H, —SO₂NR¹⁴R¹⁵, —C(═NR¹⁷)NR¹⁴R¹⁵, —N(R¹⁷)SO₂R¹⁶, and a 5 or 6-membered heterocyclic group;
or X and an adjacent R³, taken together with the atoms to which they are bonded, form an alkylenedioxy moiety;
Z is CH, CR³ or N, wherein when Z is CH or CR³, k is 0-4 and t is 0 or 1, and when Z is N, k is 0-3 and t is 0;
Y is selected from —O—, —S—, —N(R¹⁰)—, and —C(R⁴)(R⁵)—;
W¹ is selected from C₁-C₆ alkyl, C₃-C₈ cycloalkyl, aryl and Het, wherein said C₁-C₈ alkyl, C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆alkyl-C(O)SR¹⁰, —C₀-C₆ alkyl-CONR′″ R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl, is optionally unsubstituted or substituted by one or more halo substituents;
W² is selected from H, halo, C₁-C₆ alkyl, C₂-C₆ alkenyl, C₂-C₆ alkynyl, —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₀-C₆ alkyl-Ar and —C₀-C₆ alkyl-C₃-C₇ cycloalkyl, wherein said C₁-C₆ alkyl is optionally unsubstituted or substituted by one or more halo substituents, and wherein the C₃-C₇ cycloalkyl, Ar and Het moieties of said —C₀-C₆ alkyl-Het, —C₀-C₆ alkyl-Ar and —C₀-C₆ alkyl-C₃-C₇ cycloalkyl are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl, is optionally unsubstituted or substituted by one or more halo substituents;
W³ is selected from the group consisting of: H, halo, C₁-C₆ alkyl, —C₀-C₆ alkyl-NR¹¹R¹², —C₀-C₆ alkyl-SR¹⁰, —C₀-C₆ alkyl-OR¹⁰, —C₀-C₆ alkyl-C₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₁-C₆ alkyl-Ar and —C₁-C₆ alkyl-C₃-C₇ cycloalkyl, wherein said C₁-C₆ alkyl is optionally unsubstituted or substituted by one or more halo substituents;
Q is selected from C₃-C₈ cycloalkyl, Ar and Het; wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl is optionally unsubstituted or substituted by one or more halo substituents;
p is 0-8;
n is 2-8;
m is 0 or 1;
q is 0 or 1;
t is 0 or 1;
each R¹ and R² are independently selected from H, halo, C₁-C₆ alkyl, C₃-C₆ alkenyl, C₃-C₆ alkynyl, —C₀-C₆ alkyl-NR¹¹R¹², —C₀-C₆ alkyl-OR¹⁰, —C₀-C₆ alkyl-SR¹⁰, —C₁-C₆ alkyl-Het, —C₁-C₆ alkyl-Ar and —C₁-C₆ alkyl-C₃-C₇ cycloalkyl, or R¹ and R² together with the carbon to which they are attached form a 3-5 membered carbocyclic or heterocyclic ring, wherein said heterocyclic ring contains one, or more heteroatoms selected from N, O, and S, where any of said C₁-C₆ alkyl is optionally unsubstituted or substituted by one or more halo substituents;
each R³ is the same or different and is independently selected from halo, cyano, nitro, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-S₂R¹⁰, —C₀-C₆ alkyl-SOR³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, wherein said C₁-C₆ alkyl is optionally unsubstituted or substituted by one or more halo substituents;
each R⁴ and R⁵ is independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R⁶ and R⁷ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R⁸ and R⁹ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R¹⁰ is selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;
each R¹¹ and each R¹² are independently selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl, or R¹¹ and R¹² together with the nitrogen to which they are attached form a 4-7 membered heterocyclic ring which optionally contains one or more additional heteroatoms selected from N, O, and S;
R¹³ is selected from C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;
R¹⁴ and R¹⁵ are each independently selected from H, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-O—Ar, —C₀-C₆ alkyl-O-Het, —C₀-C₆ alkyl-O—C₃-C₇ cycloalkyl, —C₀-C₆ alkyl-S(O), —C₁-C₆ alkyl, —C₀-C₆ alkyl-S(O)ₓ—Ar, —C₀-C₆ alkyl-S(O)ₓ-Het, —C₀-C₆ alkyl-S(O)ₓ—C₃-C₇ cycloalkyl, —C₀-C₆ alkyl-NH-Het, —C₀-C₆ alkyl-NH—C₃-C₇ cycloalkyl, —C₀-C₆ alkyl-N(C₁-C₄ alkyl)-Ar, —C₀-C₆ alkyl-N(C₁-C₄ alkyl)-Het, —C₀-C₆ alkyl-N(C₁-C₄ alkyl)-C₃-C₇ cycloalkyl, —C₀-C₆ alkyl-Ar, —C₀-C₆ alkyl-Het and —C₀-C₆ alkyl-C₃-C₇ cycloalkyl, where x is 0, 1 or 2, or R¹⁴ and R¹⁵, together with the nitrogen to which they are attached, form a 4-7 membered heterocyclic ring which optionally contains one or more additional heteroatoms selected from N, O, and S, wherein said C₁-C₆ alkyl is optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, —CO₂H, —CO₂ (unsubstituted C₁-C₆ alkyl), —CONH₂, —CONH(unsubstituted C₁-C₆ alkyl), —CON(unsubstituted C₁-C₆ alkyl)(unsubstituted C₁-C₆ alkyl), —SO₃H, —SO₂NH₂, —SO₂NH(unsubstituted C₁-C₆ alkyl) and —SO₂N(unsubstituted C₁-C₆ alkyl)(unsubstituted C₁-C₆ alkyl);
R¹⁶ is C₁-C₆ alkyl, —C₀-C₆ alkyl-Ar or —C₀-C₆ alkyl-Het; and

R¹⁷ is H, C₁-C₆ alkyl, —C₀-C₆ alkyl-Ar or —C₀-C₆ alkyl-Het;

or a pharmaceutically acceptable salt or solvate thereof.

Compounds of formula (IV) are described in U.S. Provisional Application No. 60/368,415, filed Mar. 27, 2002: 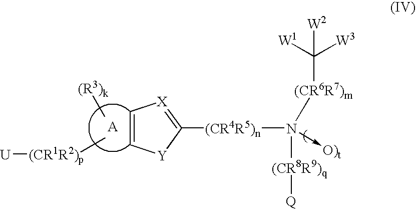
wherein:
X is CH or N;
Y is N(R¹⁰), O, or S, wherein t is 0 or 1 when Y is N(R¹⁰) or 0, and t is 0 when Y is S;
U is selected from halo, —OR¹⁰, —NR¹⁴R¹⁵, nitro, cyano, —COOR¹⁰, —COR¹³, —OCOR¹³, —CONR¹⁴R⁵, —N(R¹⁴)COR¹³, —SO₃H, —SO₂NR¹⁴R¹⁵, —C(═NR¹⁷)NR¹⁴R¹⁵, —N(R¹⁴)SO₂R¹⁶, and a 5 or 6-membered heterocyclic group;
A is a phenyl fused ring moiety or a pyridyl fused ring moiety, wherein when A is a phenyl ring moiety, k is 0-3 and t is 0 or 1 and when A is a pyridyl ring moiety, k is 0-2 and t is 0;
W¹ is selected from C₃-C₈ cycloalkyl, aryl and Het, wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₁-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR³, where said C₁-C₆ alkyl, is optionally unsubstituted or substituted by one or more halo substituents;
W² is selected from H, halo, C₁-C₆ alkyl, C₂-C₆ alkenyl, C₂-C₆ alkynyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR′″ R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₀-C₆ alkyl-Ar and —C₀-C₆ alkyl-C₃-C₇ cycloalkyl, wherein said C₁-C₆ alkyl is optionally unsubstituted or substituted by one or more halo substituents, and wherein the C₃-C₇ cycloalkyl, Ar and Het moieties of said —C₀-C₆ alkyl-Het, —C₀-C₆ alkyl-Ar and —C₀-C₆ alkyl-C₃-C₇ cycloalkyl are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR²¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said —C₁-C₆ alkyl, is optionally unsubstituted or substituted by one or more halo substituents;
W³ is selected from the group consisting of: H, halo, C₁-C₆ alkyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₁-C₆ alkyl-Ar and —C₁-C₆ alkyl-C₃-C₇ cycloalkyl, wherein said C₁-C₆ alkyl is optionally unsubstituted or substituted by one or more halo substituents;
Q is selected from C₃-C₈ cycloalkyl, Ar and Het; wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl is optionally unsubstituted or substituted by one or more halo substituents;
p is 0-8;
n is 2-8;
m is 0 or 1;
q is 0 or 1;
t is 0 or 1;
each R¹ and R² are independently selected from H, halo, C₁-C₆ alkyl, C₃-C₆ alkenyl, C₃-C₆ alkynyl, —C₀-C₆ alkyl-NR¹¹R¹², —C₀-C₆ alkyl-OR¹⁰, —C₀-C₆ alkyl-SR¹⁰, —C₁-C₆ alkyl-Het, —C₁-C₆ alkyl-Ar and —C₁-C₆ alkyl-C₃-C₇ cycloalkyl, or R¹ and R² together with the carbon to which they are attached form a 3-5 membered carbocyclic or heterocyclic ring, wherein said heterocyclic ring contains one, or more heteroatoms selected from N, O, and S, where said C₁-C₆ alkyl is optionally unsubstituted or substituted by one or more halo substituents;
each R³ is the same or different and is independently selected from halo, cyano, nitro, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, wherein said C₀-C₆ alkyl is optionally unsubstituted or substituted by one or more halo substituents;
each R⁴ and R⁵ is independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R⁶ and R⁷ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R⁸ and R⁹ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R¹⁰ is selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;
each R¹¹ and each R¹² are independently selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl, or R¹¹ and R¹² together with the nitrogen to which they are attached form a 4-7 membered heterocyclic ring which optionally contains one or more additional heteroatoms selected from N, O, and S;
R¹³ is selected from C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;
R¹⁴ and R¹⁵ are each independently selected from H, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-O—Ar, —C₀-C₆ alkyl-O-Het, —C₀-C₆ alkyl-O—C₃-C₇ cycloalkyl, —C₀-C₆ alkyl-S(O)ₓ—C₁-C₆ alkyl, —C₀-C₆ alkyl-S(O)ₓ—Ar, —C₀-C₆ alkyl-S(O)ₓ-Het, —C₀-C₆ alkyl-S(O)ₓ—C₃-C₇ cycloalkyl, —C₀-C₆ alkyl-NH—Ar, —C₀-C₆ alkyl-NH-Het, —C₀-C₆ alkyl-NH—C₃-C₇ cycloalkyl, —C₀-C₆ alkyl-N(C₁-C₄ alkyl)-Ar, —C₀-C₆ alkyl-N(C₁-C₄ alkyl)-Het, —C₀-C₆ alkyl-N(C₁-C₄ alkyl)-C₃-C₇ cycloalkyl, —C₀-C₆ alkyl-Ar, —C₀-C₆ alkyl-Het and —C₀-C₆ alkyl-C₃-C₇ cycloalkyl, where x is 0, 1 or 2, or R¹⁴ and R¹⁵, together with the nitrogen to which they are attached, form a 4-7 membered heterocyclic ring which optionally contains one or more additional heteroatoms selected from N, O, and S, wherein said C₁-C₆ alkyl is optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, —CO₂H, —CO₂ (unsubstituted C₁-C₆ alkyl), —CONH₂, —CONH(unsubstituted C₁-C₆ alkyl), —CON(unsubstituted C₁-C₆ alkyl)(unsubstituted C₁-C₆ alkyl), —SO₃H, —SO₂NH₂, —SO₂NH(unsubstituted C₁-C₆ alkyl) and —SO₂N(unsubstituted C₁-C₆ alkyl)(unsubstituted C₁-C₆ alkyl);
R¹⁶ is C₁-C₆ alkyl, —C₀-C₆ alkyl-Ar or —C₀-C₆ alkyl-Het; and
R¹⁷ is H, C₁-C₆ alkyl, —C₀-C₆ alkyl-Ar or —C₀-C₆ alkyl-Het;
or a pharmaceutically acceptable salt or solvate thereof.

Unless otherwise provided, each alkyl, alkoxy, alkenyl, alkynyl, cycloalkyl, aryl or Het (including any 3-5-membered, 4-7-membered or 5-7-membered carbocyclic or heterocyclic rings or ring moieties) in the compounds of formula (III) and (IV) is independently unsubstituted or substituted with one ore more substituents defined hereinbelow.

In the compounds of formula (IV), group A is defined as a phenyl or a pyridyl fused ring moiety and is exemplified by the following:

Group A fused ring moiety:

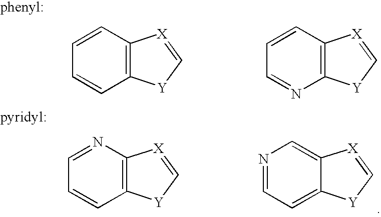

As used to define the compounds of formulas (II) or (IV), the term “alkyl” represents a straight- or branched-chain saturated hydrocarbon, containing 1 to 10 carbon atoms, unless otherwise provided, which may be unsubstituted or substituted by one or more of the substituents described below. Exemplary alkyls include, but are not limited to methyl (Me), ethyl (Et), n-propyl, isopropyl, n-butyl, isobutyl, t-butyl, n-pentyl, neopentyl and hexyl and structural isomers thereof. Any “alkyl” herein may be optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, and —CO₂H.

When combined with another substituent term as used to define the compounds of formulas (III) or (IV) (e.g., aryl or cycloalkyl as in -alkyl-Ar or -alkyl-cycloalkyl), the “alkyl” term therein refers to an alkylene moiety, that is, an unsubstituted divalent straight- or branched-chain saturated hydrocarbon moiety, containing 1 to 10 carbon atoms, unless otherwise provided. For example, the term “—C₀-C₆ alkyl-Ar”, where C is 1-6 is intended to mean the radical -alkyl-aryl (e.g., —CH₂-aryl or —CH(CH₃)-aryl) and is represented by the bonding arrangement present in a benzyl group. The term “C₀ alkyl” in a moiety, such as —C₀-C₆ alkyl-Ar or —O—(CO—C₆ alkyl)-Ar, provides for no alkyl/alkylene group being present in the moiety. Thus, when C is zero, —C₀-C₆ alkyl-Ar is equivalent to —Ar and —O—(C₀-C₆ alkyl)-Ar is equivalent to —O—Ar.

As used to define the compounds of formulas (III) or (IV), the term “alkenyl” represents a straight- or branched-chain hydrocarbon, containing 2 to 10 carbon atoms, unless otherwise provided, and one or more carbon-carbon double bonds. Alkenyl groups may be unsubstituted or substituted by one or more of the substituents described below. Exemplary alkenyls include, but are not limited ethenyl, 1-propenyl, 2-propenyl, 1-butenyl, 2-butenyl, isobutenyl, butadienyl, pentenyl and hexenyl and structural isomers thereof. Both cis (Z) and trans (E) isomers of each double bond that may be present in the compounds of formula (III) or (IV) are included within the scope of this definition. Any “alkenyl” herein may be optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, and —CO₂H.

As used to define the compounds of formulas (III) or (IV), the term “alkynyl” represents a straight- or branched-chain hydrocarbon, containing 2 to 10 carbon atoms, unless otherwise provided, and one or more carbon-carbon triple bonds and, optionally, one or more carbon-carbon double bonds. Both cis (Z) and trans (E) isomers of each double bond that may be present in the compounds of formula (III) or (IV) are included within the scope of this definition. Exemplary alkynyls include, but are not limited ethynyl, propynyl (propargyl, isopropynyl), 1-butynyl, 2-butynyl, 3-butynyl, pentynyl and hexynyl and structural isomers thereof. Any “alkynyl” herein may be optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, and —CO₂H.

As used to define the compounds of formulas (III) or (IV), when an alkenyl or alkynyl group is a substituent on an oxygen, nitrogen or sulfur atom (e.g., as in oxy (—OR), thio (—SR), ester (—CO₂R or —C(O)SR), amino (—NRR) or amido (—CONRR) moieties and the like), it is understood that a double or triple bond of the alkenyl or alkynyl group is not located on carbons that are α,β to the oxygen, nitrogen or sulfur atom. Compounds containing ene-amino or enol-type moieties (—NR—CR═CR— or —O—CR═CR—) are not intended to be included within the scope of the definition of the compounds of formula (III) or (IV).

As used to define the compounds of formulas (III) or (IV), the term “cycloalkyl” represents a non-aromatic monocyclic, bicyclic, or tricyclic hydrocarbon containing from 3 to 10 carbon atoms which may be unsubstituted or substituted by one or more of the substituents described below and may be saturated or partially unsaturated. Exemplary cycloalkyls include monocyclic rings having from 3-7, preferably 3-6, carbon atoms, such as cyclopropyl, cyclobutyl, cyclopentyl, cyclopentenyl, cyclopentadienyl, cyclohexyl, cyclohexenyl and cycloheptyl. Any “cycloalkyl” herein may be optionally substituted by one or more of the substituents independently selected from the group halo, cyano, C₁-C₆ alkyl (which specifically includes C₁-C₆ haloalkyl, —C₀-C₆ alkyl-OH, —C₀-C₆ alkyl-SH and —C₀-C₆ alkyl-NR′R″), C₃-C₆ alkenyl, oxo, —OC₁-C₆alkyl, —OC₁-C₆ alkenyl, —C₀-C₆ alkyl-COR′, —C₀-C₆ alkyl-CO₂R′, —C₀-C₆ alkyl-CONR′R″, —OC₀-C₆ alkyl-CO₂H, —OC₂-C₆ alkyl-NR′R″, and —C₀-C₆ alkyl-SO₂NR′R″, wherein each R′ and R″ are independently selected from H or unsubstituted C₁-C₆ alkyl.

As used to define the compounds of formulas (III) or (IV), the terms “Ar” or “aryl” is used interchangeably at all occurrences mean a substituted or unsubstituted carbocyclic aromatic group, which may be optionally fused to another carbocyclic aromatic group moiety or to a cycloalkyl group moiety, which may be optionally substituted or unsubstituted. Examples of suitable Ar or aryl groups include phenyl, naphthyl indenyl, 1-oxo-1H-indenyl and tetrahydronaphthyl. Any “Ar”, “aryl” or “phenyl” herein may be optionally unsubstituted or substituted by one or more of the substituents independently selected from the group halo, cyano, C₁-C₆ alkyl (which specifically includes C₁-C₆ haloalkyl, —C₀-C₆ alkyl-OH, —C₀-C₆ alkyl-SH and —C₀-C₆ alkyl-NR′R″), C₃-C₆ alkenyl, —OC₁-C₆alkyl, —OC₁-C₆ alkenyl, —C₀-C₆ alkyl-COR′, —C₀-C₆ alkyl-CO₂R′, —C₀-C₆ alkyl-CONR′R″, —OC₀-C₆ alkyl-CO₂H, —OC₂₋₇C₆ alkyl-NR′R″, —C₀-C₆ alkyl-C(═NR′)NR′R″, and —C₀-C₆ alkyl-SO₂NR′R″, wherein each R′ and R″ are independently selected from H or unsubstituted C₁-C₆ alkyl.

As used to define the compounds of formulas (III) or (IV), the term “Het” means a stable 5- to 7-membered monocyclic, a stable 7- to 10-membered bicyclic, or a stable 11- to 18-membered tricyclic heterocyclic ring group, all of which are saturated, unsaturated or aromatic, and consist of carbon atoms and from one to three heteroatoms selected from the group consisting of N, O and S, and which includes bicyclic and tricyclic rings containing one or more fused cycloalkyl, aryl (e.g., phenyl) or heteroaryl (aromatic Het) ring moieties. As used herein the term “Het” is also intended to encompass heterocyclic groups containing nitrogen and/or sulfur where the nitrogen or sulfur heteroatoms are optionally oxidized or the nitrogen heteroatom is optionally quaternized. The heterocyclic group may be attached at any heteroatom or carbon atom that results in the creation of a stable structure. Any “Het” herein may be optionally unsubstituted or substituted by one or more of the substituents independently selected from the group halo, cyano, C₁-C₆ alkyl (which specifically includes C₁-C₆ haloalkyl, —C₀-C₆ alkyl-OH, —C₀-C₆ alkyl-SH and —C₀-C₆ alkyl-NR′R″), C₃-C₆ alkenyl, oxo, —OC₁-C₆alkyl, —OC₁-C₆ alkenyl, —C₀-C₆ alkyl-COR′, —C₀-C₆ alkyl-CO₂R′, —C₀-C₆ alkyl-CONR′R″, —OC₀-C₆ alkyl-CO₂H, —OC₂-C₆ alkyl-NR′R″, —C₀-C₆ alkyl-C(═NR′)NR′R″ and —C₀-C₆ alkyl-SO₂NR′R″, wherein each R′ and R″ are independently selected from H or unsubstituted C₁-C₆ alkyl.

Examples of such heterocyclic groups include, but are not limited to piperidinyl, piperazinyl, 2-oxopiperazinyl, 2-oxopiperidinyl, 2-oxopyrrolodinyl, 2-oxoazepinyl, azepanyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, 1,3-benzodioxolyl (e.g., methylenedioxy-substituted phenyl), 1,4-benzodioxolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, benzofuranyl, benzothienyl, dihydrobenzofuranyl, dihydrobenzothienyl, dihydroindolyl, tetrazolyl, thiamorpholinyl sulfoxide, thiamorpholinyl sulfone, and oxadiazolyl, as well as triazolyl, thiadiazolyl, oxadiazolyl, isoxazolyl, isothiazolyl, imidazolyl, pyridazinyl, pyrimidinyl and triazinyl which are available by routine chemical synthesis and are stable.

Examples of the 4-7 membered heterocyclic rings useful in the compounds of formula (III) or (IV), include, but are not limited to azetidinyl, piperidinyl, piperazinyl, 2-oxopiperazinyl, 2-oxopiperidinyl, 2-oxopyrrolodinyl, azepanyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, furyl, pyranyl, tetrahydrofuryl, tetrahydropyranyl, thienyl, tetrazolyl, thiamorpholinyl sulfoxide, thiamorpholinyl sulfone, and oxadiazolyl, as well as triazolyl, thiadiazolyl, oxadiazolyl, isoxazolyl, isothiazolyl, imidazolyl, pyridazinyl, pyrimidinyl and triazinyl which are available by routine chemical synthesis and are stable. The 4-7 membered heterocyclic group may be optionally unsubstituted or substituted by one or more of the substituents independently selected from the group halo, cyano, C₁-C₆ alkyl (which specifically includes C₁-C₆ haloalkyl, —C₀-C₆ alkyl-OH, —C₀-C₆ alkyl-SH and —C₀-C₆ alkyl-NR′R″), C₃-C₆ alkenyl, oxo, —OC₁-C₆alkyl, —OC₁-C₆ alkenyl, —C₀-C₆ alkyl-COR′, —C₀-C₆ alkyl-CO₂R′, —C₀-C₆ alkyl-CONR′R″, —OC₀-C₆ alkyl-CO₂H, —OC₂-C₆ alkyl-NR′R″, —C₀-C₆ alkyl-C(═NR′)NR′R″ and —C₀-C₆ alkyl-SO₂NR′R″, wherein each R′ and R″ are independently selected from H or unsubstituted C₁-C₆ alkyl.

Examples of 5 or 6 membered heterocyclic groups include, but are not limited to piperidinyl, piperazinyl, 2-oxopiperazinyl, 2-oxopiperidinyl, 2-oxopyrrolodinyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, furyl, pyranyl, tetrahydrofuryl, tetrahydropyranyl, thienyl, tetrazolyl, thiamorpholinyl sulfoxide, thiamorpholinyl sulfone, and oxadiazolyl, as well as triazolyl, thiadiazolyl, oxadiazolyl, isoxazolyl, isothiazolyl, imidazolyl, pyridazinyl, pyrimidinyl and triazinyl which are available by routine chemical synthesis and are stable. The 5-6 membered heterocyclic group may be attached at any heteroatom or carbon atom that results in the creation of a stable structure. The 5-6 membered heterocyclic group may be optionally unsubstituted or substituted by one or more of the substituents independently selected from the group halo, cyano, C₁-C₆ alkyl (which specifically includes C₁-C₆ haloalkyl, —C₀-C₆ alkyl-OH, —C₀-C₆ alkyl-SH and —C₀-C₆ alkyl-NR′R″), C₃-C₆ alkenyl, oxo, —OC₁-C₆alkyl, —OC₁-C₆ alkenyl, —C₀-C₆ alkyl-COR′, —C₀-C₆ alkyl-CO₂R′, —C₀-C₆ alkyl-CONR′R″, —OC₀-C₆ alkyl-CO₂H, —OC₂-C₆ alkyl-NR′R″, —C₀-C₆ alkyl-C(═NR′)NR′R″ and —C₀-C₆ alkyl-SO₂NR′R″, wherein each R′ and R″ are independently selected from H or unsubstituted C₁-C₆ alkyl.

In the compounds of formulas (III) and (IV), the terms “halogen” and “halo” represent chloro, fluoro, bromo or iodo substituents; “alkoxy” is intended to mean the radical —ORₐ, where Rₐ is an alkyl group, wherein alkyl is as defined above, provided that —O—C₁ alkyl may be optionally substituted by one or more of the substituents independently selected from the group halo and —CO₂H. (exemplary alkoxy groups include methoxy, ethoxy, propoxy, and the like); “phenoxy” is intended to mean the radical —ORₐᵣ, where Rₐᵣ is a phenyl group; “acetoxy” is intended to mean the radical —O—C(═O)-methyl; “benzoyloxy” is intended to mean the radical —O—C(═O)-phenyl; and “oxo” is intended to mean the keto diradical ═O, such as present on a pyrrolidin-2-one ring.

A method for the preparation of compounds of formula (III), comprises the steps of:
(a) reacting an alcohol having the formula: HY′—(CR⁴R⁵)ₙ-L, where Y′ is —O—, —S—, —NH or protected —NH and L is a leaving group, such as a halogen (iodide, bromide or chloride), sulfonate (tosylate, mesylate, triflate, etc.) or is a group that is converted to a leaving group (e.g., an alcohol), with an alcohol having the formula: 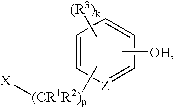
   where X is a protected carboxylic acid moiety, to form a compound having the formula: 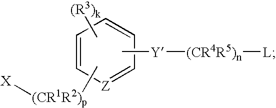
(b) reacting the compound formed in step (a) with a secondary amine having the formula 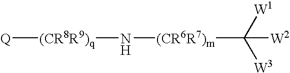
   to form a compound having the formula: 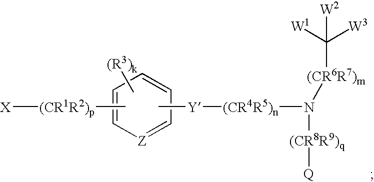
(c) converting the protected carboxylic acid moiety into a desired amide moiety; and
(d) optionally oxidizing the compound formed in step (b) to the N-oxide thereof.

Another method for the preparation of compounds of formula (III), comprises the steps of:
(a) reacting an acetylene having the formula: R′O—(CR⁴R⁵)ₙ₋₁—C≡C—H, where R′ is a hydroxyl protecting group, with a halogen-containing aromatic compound having the formula 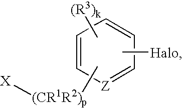
   where X is a protected carboxylic acid moiety and Halo is bromo or iodo, in the presence of a catalyst to form a compound having the formula: 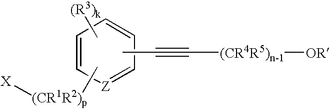
(b) reducing the compound formed in step (a) and converting the protected hydroxyl group into a leaving group, L, such as a halogen (iodide, bromide or chloride), sulfonate (tosylate, mesylate, triflate, etc.) or is a group that is converted to a leaving group (e.g., an alcohol), to form a compound having the formula: 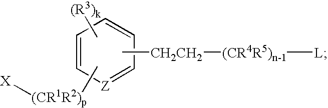
(c) reacting the compound formed in step (b) with an amine having the formula: 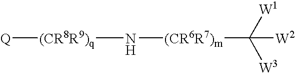
   to form a compound having the formula: 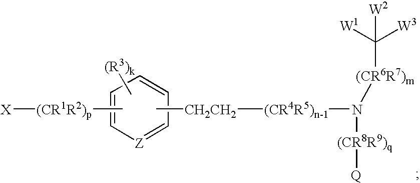
   (d) converting the protected carboxylic acid moiety into a desired amide moiety; and
(e) optionally oxidizing the compound formed in step (b) to the N-oxide thereof.

Another method for the preparation of compounds of formula (III), comprises the steps of:
(a) reacting an alcohol having the formula: L′-(CR⁴R⁵)ₙ-L, where L′ and L are leaving groups, which may be the same or different, such as a halogen (iodide, bromide or chloride), sulfonate (tosylate, mesylate, triflate, etc.) or is a group that is converted to a leaving group (e.g., an alcohol), with a compound having the formula: 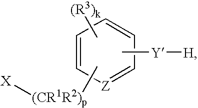
   where Y′ is —O—, —S—, or —NH— and X is defined as above or a protected form thereof, to form a compound having the formula: 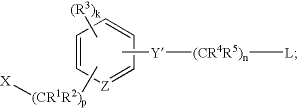
(b) reacting the compound formed in step (a) with a secondary amine having the formula 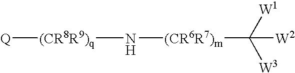
   to form a compound having the formula: 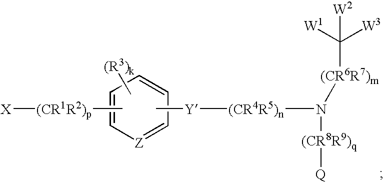
(c) removing any protecting groups; and
(d) optionally oxidizing the compound formed in step (b) or (c) to the N-oxide thereof.

Another method for the preparation of compounds of formula (III), comprises the steps of:
(a) reacting a compound having the formula: 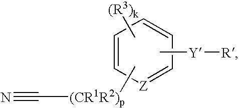
   where Y′ is —O—, —S—, or —NH— and R′ is a suitable protecting group for —OH, —SH, or —NH₂, with a hydrazide or azide to form a heterocyclic-containing compound having the formula: 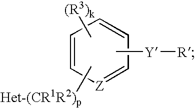
(b) optionally protecting the NH moiety of the heterocyclic group with a protecting group, and removing the R′ protecting group;
(c) reacting the compound formed in step (b) with a compound having the formula: L′-(CR⁴R⁵)ₙ-L, where L′ and L are leaving groups, which may be the same or different, such as a halogen (iodide, bromide or chloride), sulfonate (tosylate, mesylate, triflate, etc.) or is a group that is converted to a leaving group (e.g., an alcohol), to form a compound having the formula: 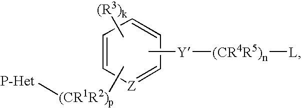
   where P is an optional protecting group or H;
(d) reacting the compound formed in step (c) with an amine having the formula: 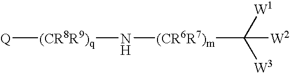
   to form a compound having the structure: 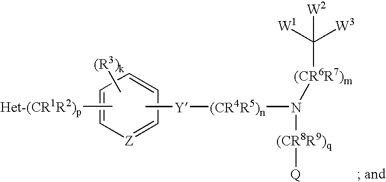
(e) removing any protecting groups.

Another method for the preparation of compounds of formula (III), comprises the steps of:
(a) reacting an acetylene having the formula: R′O—(CR⁴R⁵)ₙ₋₁—C≡C—H, where R′ is a hydroxyl protecting group, with a halogen-containing aromatic compound having the formula 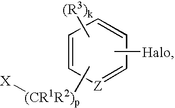
   where Halo is bromo or iodo, in the presence of a catalyst to form a compound having the formula: 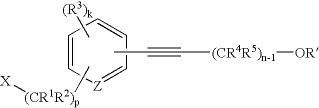
(b) reducing the compound formed in step (a) and converting the protected hydroxyl group into a leaving group, L, such as a halogen (iodide, bromide or chloride), sulfonate (tosylate, mesylate, triflate, etc.) or is a group that is converted to a leaving group (e.g., an alcohol) to form a compound having the formula: 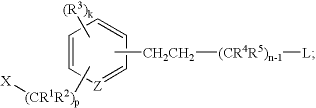
(c) reacting the compound formed in step (b) with an amine having the formula: 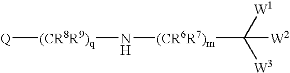
   to form a compound having the formula: 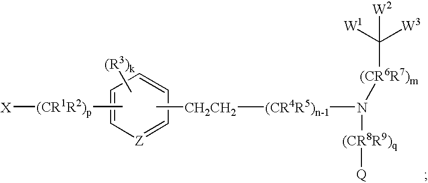
(d) removing any protecting groups; and
(e) optionally oxidizing the compound formed in step (c) or (d) to the N-oxide thereof.

Another method for the preparation of compounds of formula (III), comprises the steps of:
(a) reacting an alcohol having the formula: HO—(CR⁴R⁵)ₙ-L, where L is a leaving group, such as a halogen (iodide, bromide or chloride), sulfonate (tosylate, mesylate, triflate, etc.) or is a group that is converted to a leaving group (e.g., an alcohol) with a phenol having the formula: 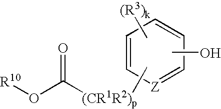
   to form an aryl ether having the formula: 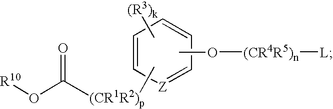
(b) reacting an amine having the formula 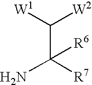
   with and an aldehyde having the formula Q-CHO or a ketone to form a secondary amine having the formula: 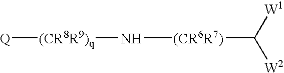
(c) reacting the ether formed in step (a) with the secondary amine formed in step (b) to form a compound of this invention having the formula: 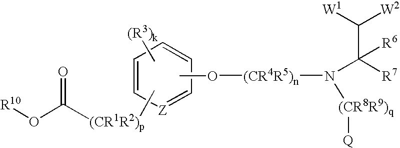
(d) when R¹⁰ is other than H, optionally converting the compound formed in step (c) to the compound of this invention, wherein R¹⁰ is H.

Another method for the preparation of compounds of formula (III), comprises the steps of:
(a) reacting an alcohol having the formula: HO—(CR⁴R⁵)ₙ-L, where L is a leaving group, such as a halogen (iodide, bromide or chloride), sulfonate (tosylate, mesylate, triflate, etc.) or is a group that is converted to a leaving group (e.g., an alcohol), with an amine having the formula: 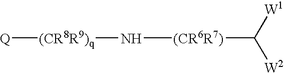
   to form a tertiary amine having the formula: 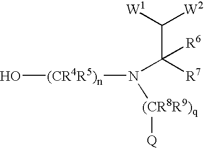
(b) reacting the tertiary amine formed in step (a) with a phenol having the formula: 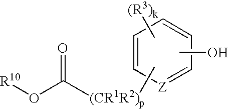
   to form a compound of this invention having the formula: 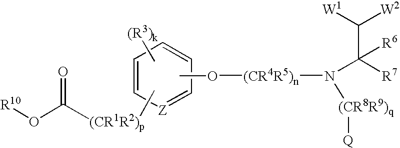
(c) when R¹⁰ is other than H, optionally converting the compound formed in step (b) to the compound of this invention, wherein R¹⁰ is H.

Another method for the preparation of compounds of formula (III), comprises the steps of:
(a) reacting an alcohol having the formula: HO—(CR⁴R⁵)ₙ-L, where L is a leaving group, such as a halogen (iodide, bromide or chloride) or sulfonate (tosylate, mesylate, triflate, etc.), with a phenol having the formula: 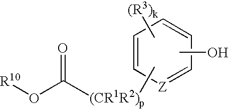
   to form an ether-alcohol having the formula: 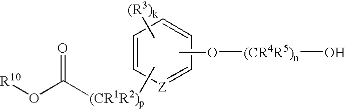
(b) converting alcohol moiety of the ether-alcohol formed in step (a) into L′, where L′ is a leaving group such as a halogen (iodide, bromide or chloride), sulfonate (tosylate, mesylate, triflate, etc.) or is a group that is converted to a leaving group (e.g., an alcohol) and treating the resulting compound with an amine having the formula: 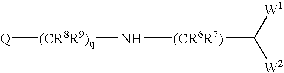
   to form a compound of this invention having the formula: 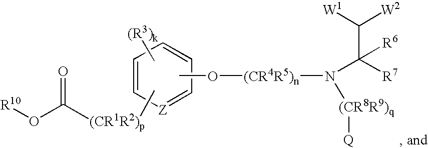
(c) when R¹⁰ is other than H, optionally converting the compound formed in step (b) to the compound of this invention, wherein R¹⁰ is H.

The method for the preparation of compounds of formula (IV), comprises the steps of:
(a) coupling an acetylene having the formula: with a phenol having the formula: 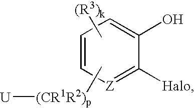
   where Halo is a halogen selected from iodo or bromo, in the presence of a metal catalyst to form an aryl-alcohol having the formula: 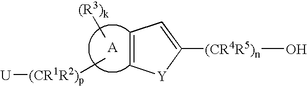
(b) converting alcohol moiety of the aryl-alcohol formed in step (a) into L′, where L′ is a leaving group such as a halogen (iodide, bromide or chloride), sulfonate (tosylate, mesylate, triflate, etc.) or is a group that is converted to a leaving group (e.g., an alcohol), and treating the resulting compound with an amine having the formula: 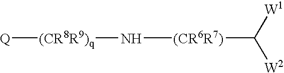
   to form the compound of formula (IV);
(c) optionally converting the compound of formula (IV) from step (b) into another compound of formula (IV); and
(d) optionally oxidizing the compound formed in step (c) to the N-oxide thereof.

Alternatively, the compounds of formula (IV) may be prepared by
(a) coupling an acetylene having the formula: with a phenol having the formula: 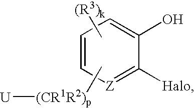
   where Halo is a halogen selected from iodo or bromo, in the presence of a metal catalyst to form an aryl-alcohol having the formula: 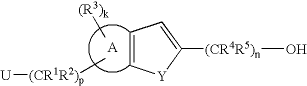
(b) converting alcohol moiety of the aryl-alcohol formed in step (a) into L′, where L′ is a leaving group such as a halogen (iodide, bromide or chloride) or a sulfonate (tosylate, mesylate, triflate, etc.) and treating the resulting compound with sodium azide, followed by hydrogenation in the presence of a palladium catalyst to form a primary amine having the formula: 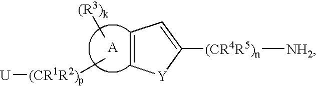
(c) treating the primary amine with a first aldehyde in the presence of a reducing agent, to form a secondary amine and treating the secondary amine with a second aldehyde in the presence of a reducing agent to form the compound of formula (IV); 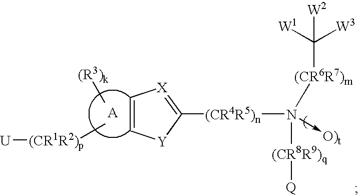
(d) optionally converting the compound of formula (IV) from step (b) into another compound of formula (IV); and
(e) optionally oxidizing the compound formed in step (b) or (c) to the N-oxide thereof.

International Patent Application WO 01/41704 (Merck & Co., Inc.) discloses a compound of formula (V): 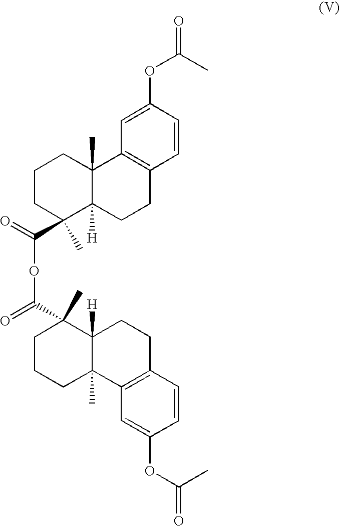
the use of compound (VI): 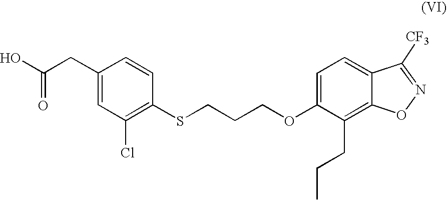
and related compounds alongside methods for their production as described in International Patent Application WO97/28137 (Merck & Co., Inc), along with methods for making them, as being useful as an agonist of LXR and their use in pharmaceutical formulations to reverse cholesterol transport and treat atherosclerotic cardiovascular diseases and related diseases.

Other LXR agonists may be identified by assays such as those described in the above referenced patent applications, for example, the assays described in Examples 1 and 2 of PCT/US01/27622. Biotinylated LXRβ protein was incubated for 20-25 minutes at a concentration of 25 nM in assay buffer (50 mM KCl, 50 mM Tris-pH8, 0.1 mg/ml FAF-BSA, 10 mM DTT) with equimolar amounts of streptavidin-AlloPhycoCyanin (APC, Molecular Probes). At the same time, the biotinylated peptide comprising amino acids 675-699 of SRC-1 (CPSSHSSLTERHKILHRLLQEGSPS-CONH2) (SEQ ID No. 2) at a concentration of 25 nM was incubated in assay buffer with a ½ molar amount of streptavidin-labelled Europium (Wallac) for 20-25 minutes. After the initial incubations are completed, a 10 molar excess (250 nM) of cold biotin was added to each of the solutions to block the unattached streptavidin reagents. After 20 min at room temp, the solutions were mixed yielding a concentration of 12.5 nM for the dye-labelled LXRβ protein and SRC-1 peptide. 80 μL of the protein/peptide mixture was added to each well of an assay plate containing 20 μL of test compound. The final volume in each well was 0.1 mL, and the concentration in the well for the dye-labelled protein and peptide was 10 nM. The final test compound concentrations were between 56 pM and 10 μM. The plates were incubated at room temp in the dark for 4-12 hours and then counted on a Wallac Victor fluorescent plate reader. In this assay 1 μM 24(S), 25-epoxycholesterol gave a reading of 20000 fluorescence units over a background reading of 10000 fluorescence units. The assay for LXRα was run according to the procedures described above using his-tagged LXRα ligand binding domain (amino acids 183-447 of Genbank accession number U22662, with the 14^{th} amino acid corrected to A from R).

The invention provides the use of a LXR agonist in the preparation of a medicament for the treatment and/or prophylaxis of diseases or conditions characterised by neuron degeneration, inflammation in the CNS, injury or impaired plasticity.

The invention also provides a method of treating or preventing diseases or disorders characterised by neuron degeneration, inflammation in the CNS, injury or impaired plasticity which comprises administering to a subject in need thereof an effective non-toxic and pharmaceutically acceptable amount of a LXR agonist, such as compounds of formula (I), (II), (III), (IV), (V) and (VI) or a pharmaceutically acceptable derivative thereof.

Furthermore, the invention provides the use of a LXR agonist in the preparation of a medicament for the promotion of growth and/or repair of neurons in diseases or conditions characterised by neuron degeneration, inflammation in the CNS, injury or impaired plasticity which method comprises the administration of an effective, non-toxic and pharmaceutically acceptable amount of a LXR agonist, such as compounds of formula (I), (II), (III), (IV), (V) and (VI) or a pharmaceutically acceptable derivative thereof.

The invention also provides a method for the promotion of growth and/or repair of neurons in diseases or conditions characterised by neuron degeneration, inflammation in the CNS, injury or impaired plasticity which method comprises the administration of an effective, non-toxic and pharmaceutically acceptable amount of a LXR agonist, such as compounds of formula (I), (II), (III), (IV), (V) and (VI) or a pharmaceutically acceptable derivative thereof.

Suitable diseases or conditions are those characterised by neuron degeneration.

Suitable diseases or conditions are those characterised by neuron injury.

Suitable diseases or conditions are those characterised by impaired plasticity.

Suitable diseases or conditions are those characterised by inflammation in the CNS.

Particular diseases or conditions are characterised by neuron degeneration and inflammation, and thus benefiting from the growth and/or repair of neurons including stroke, Alzheimer's disease, fronto-temporal dementias (tauopathies), peripheral neuropathy, Parkinson's disease, dementia with Lewy bodies, Huntington's disease, amyotrophic lateral sclerosis and multiple sclerosis.

Diseases or conditions characterised by neuron degeneration and/or impaired plasticity include psychiatric disorders such as schizophrenia and depression.

Particular diseases or conditions characterised by neuronal injury include those conditions associated with brain and/or spinal cord injury, including trauma.

Accordingly, the present invention also provides a pharmaceutical composition for the promotion of growth and/or repair of neurons in diseases or conditions characterised by neuron degeneration, inflammation in the CNS, injury or impaired plasticity, which composition comprises a LXR agonist and a pharmaceutically acceptable carrier therefor.

Suitable pharmaceutically acceptable salts include salts of salts derived from appropriate acids, such as acid addition salts, or bases.

Suitable pharmaceutically acceptable salts include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-b-phenethylamine, dehydroabietylamine, N,N′-bisdehydroabietylamine, glucamine, N-methylglucamine or bases of the pyridine type such as pyridine, collidine, quinine or quinoline.

Suitable acid addition salts include pharmaceutically acceptable inorganic salts such as the sulphate, nitrate, phosphate, borate, hydrochloride and hydrobromide and pharmaceutically acceptable organic acid addition salts such as acetate, tartrate, maleate, citrate, succinate, benzoate, ascorbate, methane-sulphonate, a-keto glutarate and a-glycerophosphate.

The LXR agonists referred to herein are conveniently prepared according to the methods disclosed in the above mentioned patent publications in which they are disclosed.

The salts and/or solvates of the LXR agonists may be prepared and isolated according to conventional procedures for example those disclosed in the, above mentioned, patent publications.

In the above mentioned method the LXR agonist, may be administered per se or, preferably, as a pharmaceutical composition also comprising a pharmaceutically acceptable carrier.

In the treatment of the invention, the LXR agonist mentioned herein is formulated and administered in accordance with the methods disclosed in the above mentioned patent applications and patents.

As used herein the term ‘pharmaceutically acceptable’ embraces compounds, compositions and ingredients for both human and veterinary use: for example the term ‘pharmaceutically acceptable salt’ embraces a veterinarily acceptable salt.

The composition may, if desired, be in the form of a pack accompanied by written or printed instructions for use.

Usually the pharmaceutical compositions of the present invention will be adapted for oral administration, although compositions for administration by other routes, such as by injection and percutaneous absorption are also envisaged.

Particularly suitable compositions for oral administration are unit dosage forms such as tablets and capsules. Other fixed unit dosage forms, such as powders presented in sachets, may also be used.

In accordance with conventional pharmaceutical practice, the carrier may comprise a diluent, filler, disintegrant, wetting agent, lubricant, colourant, flavourant or other conventional adjuvant.

Typical carriers include, for example, microcrystalline cellulose, starch, sodium starch glycollate, polyvinylpyrrolidone, polyvinylpolypyrrolidone, magnesium stearate, sodium lauryl sulphate or sucrose.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Compositions may contain from 0.1% to 99% by weight, preferably from 10-60% by weight, of the active material, depending upon the method of administration.

Compositions may, if desired, be in the form of a pack accompanied by written or printed instructions for use.

The compositions are formulated according to conventional methods, such as those disclosed in standard reference texts, for example the British and US Pharmacopoeias, Remington's Pharmaceutical Sciences (Mack Publishing Co.), Martindale The Extra Pharmacopoeia (London, The Pharmaceutical Press) and Harry's Cosmeticology (Leonard Hill Books).

One index of synaptic plasticity is increased synaptic transmission. This can be measured in cultured hippocampal neurons using electrophysiological recordings as described by Levine E S, Crozier R A, Black I B, Plummer M R. “Brain derived neurotrophic factor modulates hippocampal synaptic transmission by increasing N-methyl-D aspartic acid receptor activity”, in Proc. Natl. Acad. Sci USA Vol 95 pp10235-10239 (1998). Thus the neurons would be treated with the compound under test and then their synaptic transmission determined against a control following glutamate exposure.

No adverse toxicological effects are expected for the compositions or methods of the invention in the above mentioned dosage ranges.

Although the central nervous system (CNS) accounts for <10% of total body mass, it contains roughly a quarter of all the unesterified cholesterol present in the body (29). Virtually all of the cholesterol present in the brain is derived from in situ biosynthesis. The conversion of cholesterol to the LXR ligand 24(S)-hydroxycholesterol, which can cross the blood brain barrier (BBB) and enter the general circulation, represents an important mechanism for cholesterol flux out of the CNS (30-32). Importantly, the dysregulation of cholesterol balance in the brain may be related to the onset of neurological disease (29). Cholesterol turnover across the brain is increased in neurodegenerative disorders such as Alzheimer's disease (AD) and Niemann-Pick Type C disease (33-34). Moreover, there is clinical evidence that patients with elevated cholesterol levels have increase susceptibility to AD (35, 36), and, conversely, that treatment with the statin class of cholesterol-lowering drugs reduces the incidence of AD (37,38). Finally, the E2 and E4 isoforms of apoE, which transports cholesterol throughout the body, have been genetically linked to either a decreased or increased risk of AD, respectively (39-41). Thus, understanding the mechanisms regulating cholesterol balance in the brain may provide important insights into the etiology and treatment of neurodegenerative disorders.

In recent years, great strides have been made in understanding the functions of LXRα and LXRβ in the regulation of cholesterol homeostasis. The LXRs regulate a number of genes involved in the biosynthesis, transport, and excretion of cholesterol and thus are likely to have important implications in human diseases such as hypercholesterolemia and atherosclerosis (25). However, the potential role that the LXRs might play in the CNS has remained largely undefined. The brain is the most cholesterol-rich organ in the body, and dysregulation of cholesterol homeostasis may influence the neurological disorders such as AD (35-38, 42, 43). The brain also produces virtually all of the body's 24(S)-hydroxycholesterol, a cholesterol metabolite that serves as an efficacious agonist of both LXR subtypes (27, 28, 30). The expression patterns of cholesterol-24-hydroxylase, the enzyme that synthesizes 24(S)-hydroxycholesterol, and LXRβ within the CNS are remarkably similar (26, 44). These observations suggest that the LXRs might serve as integral components of a regulatory loop that modulates cholesterol levels and/or cholesterol partitioning in the brain.

In summary, in Examples 6-11 below, the inventors of the present invention demonstrate that LXR regulates a series of genes involved in cholesterol homeostasis in the CNS, both in vitro and in vivo, as well as cholesterol efflux from cultured astroglial cells. There is mounting evidence that cholesterol balance has an important impact on the onset and/or progression of various CNS disorders, including AD. Thus, it is believed that LXR ligands and agonists will have utility in the treatment of a range of CNS disorders caused by either trauma or disease, including AD.

The following Examples are intended for illustration only and are not intended to limit the scope of the invention in any way; the present invention being defined by the appended claims.

### EXAMPLES

### Example 1

### 2-(3-{3-[[2-Chloro-3-(trifluoromethyl)benzyl](2,2-diphenylethyl)amino]propoxy}-phenyl)acetic Acid

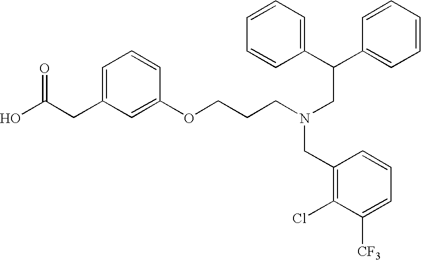

Argogel-MB-OH (6.0 g, 2.40 mmol, Argonaut Technologies) was treated with a solution of (3-{[tert-butyl(dimethyl)silyl]oxy}phenyl)acetic acid (5.40 g, 19.2 mmol, Eur. Pat. Appl. (1987) Application: EP 87-303742 19870428) in 50 mL of anhydrous dichloromethane followed by dicyclohexylcarbodiimide (4.16 g, 19.2 mmol) and 4-dimethylaminopyridine (2.50 g, 19.2 mmol). After rotating at room temperature for 15 hours, the resin was filtered, washed sequentially with dichloromethane (2×25 mL), dimethylformamide (2×25 mL), dichloromethane (3×25 mL), methanol (3×25 mL), dichloromethane (3×25 mL) and diethyl ether (2×25 mL). After drying under house vacuum overnight at 40° C., the resin was treated with 1.0 M tetrabutylammonium fluoride (24 mL, 23.4 mmol) in tetrahydrofuran, and the mixture was rotated for 4 hours. The resin was filtered, washed sequentially with dichloromethane (2×25 mL), dimethylformamide (2×25 mL), dichloromethane (3×25 mL), methanol (3×25 mL), and dichloromethane (3×25 mL) to give the deprotected phenol. The dry resin was treated with 90 mL of anhydrous toluene followed by triphenylphosphine (15.8 g, 60.0 mmol) and 3-bromo-1-propanol (8.4 g, 60.0 mmol). Upon cooling to 0° C., diisopropyl azodicarboxylate (12.1 g, 60.0 mmol) in 20 mL of anhydrous toluene was added in a dropwise fashion. The reaction was allowed to warm to room temperature and stirred for 15 hours. The resin was filtered, washed sequentially with dichloromethane (2×50 mL), dimethylformamide (2×50 mL), dichloromethane (3×50 mL), methanol (2×50 mL) and dichloromethane (3×50 mL), and dried under house vacuum. The bromide functionalized resin was treated with a solution of diphenethylamine (25.0 g, 127 mmol) in 60 mL of anhydrous dimethylsulfoxide, and the reaction was rotated for 15 hours. The resin was filtered, washed sequentially with dichloromethane (2×50 mL), dimethylformamide (2×50 mL), dichloromethane (3×50 mL), methanol (3×50 mL) and dichloromethane (3×50 mL), and dried under house vacuum at 40° C. The secondary amine resin (5.75 g, 2.0 mmol) was treated with a solution of 2-chloro-3-trifluoromethylbenzaldehyde (8.32 g, 40.0 mmol) in 80 mL of 8% acetic acid in dimethylformamide. Solid sodium triacetoxyborohydride (8.5 g, 40.0 mmol) was added, and the reaction was rotated for 15 hours. The resin was filtered, washed sequentially with dichloromethane (2×50 mL), dimethylformamide (2×50 mL), dichloromethane (3×50 mL), methanol (3×50 mL) and dichloromethane (3×50 mL), and dried under house vacuum overnight at 50° C. The resin-bound product was treated with 30 mL of trifluoroacetic acid/dichloromethane (15/85) for 15 minutes, and the filtrate was collected. The cleavage procedure was repeated again, and the combined filtrates were concentrated under reduced pressure. The crude product was purified by preparative thin layer chromatography (silica gel, 1 mm plates, Merck 20×20 cm silica gel 60 F₂₅₄) eluting with methanol:dichloromethane (3:97) to give 7.0 mg of the title compound (5% yield based on theoretical loading of secondary amine resin) of a viscous oil: ¹H NMR (CDCl₃, 400 MHz) δ 7.42 (d, 1H, J=7.6), 7.23-7.10 (m, 12H), 6.85 (t, 2H, J=8.1), 6.63 (s, 1H), 6.61 (s, 1H), 4.11 (t, 1H, J=7.8), 3.75 (s, 2H), 3.63 (t, 2H, J=6.0), 3.59 (s, 2H), 2.12 (d, 2H, J=7.8), 2.67 (t, 2H, J=6.6), 1.81 (tt, 2H, J=6.2); MS (ESP+) m/e 582 (MH⁺); TLC (EtOAc:hexanes/1:1) R_{f}=0.58.

### Example 2

### N-(2,2,2-trifluoroethyl)-N-[4-(2,2,2-trifluoro-1-hydroxy-1-trifluoromethyl-ethyl)-phenyl]-benzenesulfonamide (T0901317)

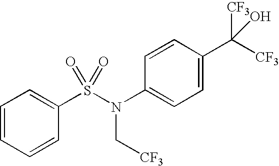 12.1 Preparation of N-trifluoroethylaniline Derivative. 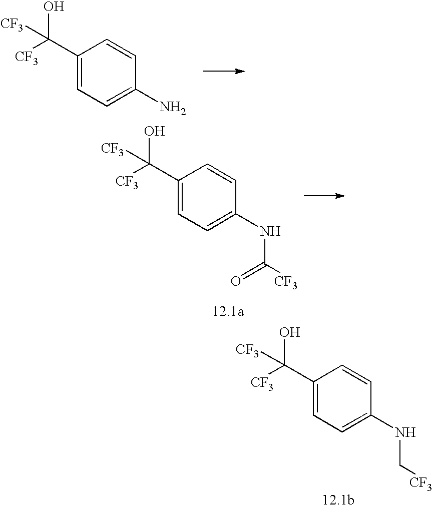
To a suspension of 4-[2,2,2-triflouro-1-hydroxy-1-(trifluoromethyl)ethyl]aniline (9.07 g, 35.0 mmol) in CH2CL2 (100 ml) was added to solution of trifluoroacetic anhydride (5.7 ml, 40.2 mmol) in CH2Cl2 (50 ml) dropwise at room temperature. The solution was stirred for 3 hours, the solution cleared and TLC indicated that the reaction was completed. The reaction mixture was washed with water, aqueous NaHCO3, and brine. The organic layer was drawn off, dried over MgSO4, filtered and concentrated to give 12.1 g of the intermediate trifluoroacetanitrilide (12.1a). The intermediate 12.1a was taken up in the THF (50 ml) and treated with LiAlH4 (4.00 g, 106 mmol) at refklux for 10 hours. The reaction was quenched sequentially adding 4 ml of water, 4 ml of 15% NaOH and 12 ml of water. The resulting suspension was stirred for an additional 30 minutes, filtered through a celite pad, which was then rinsed with THF. The combined filtrate and rinse was concentrated under reduced pressure. The residue was taken up in EtOAc, washed with Brine, dried over MgSO4, filtered and concentrated. The resulting crude product was purified by chromatography on SiO2 (4:1 hexane:EtOAc as eluant) to provide 11.og (92%) of the title compound (12.1b).

¹H NMR (CDCl₃): δ 7.52 (J=8.6 Hz, 2H), 6.72 (d, J=8.6 Hz, 2H), 4.10 (bs, 1H), 3.80 (q, J=8.5 Hz, 2H), 3.31 (bs, 1H). MS (ES+): 342 (M+H, 100).

12.2 Sulfonylation of 12.1b

A sample of 12.1b from above (1.87 g, 5.48 mmol) was treated with benzenesulfonylchloride (1.18 g, 6.68 mmol) in pyridine (10 ml) at room temperature for 10 days. The reaction mixture as diluted with EtOAc, washed with aqueous NaHCO3, and brine. The organic layer was dried over MgSO4, filtered and concentrated. The crude product was purified by chromatography on SiO2 (4:1 hexane:EtOAc as eluant) to provide 1.65 g (62%) of compound 12.

¹H NMR (CDCl₃): δ 7.78 (J=8.8 Hz, 2H), 7.61 (t, J=7.6 Hz, 1H), 7.58 (d, J=7.6 Hz, 2H), 7.46 (t, J=Hz, 2H), 4.24 (q, J=8.2 Hz, 2H), 3.41 (s, 1H). MS (ES−): 480 (M−H, 100). Anal. Calcd. for C₁₇H₁₂F₉NO₃S:C, 42.42; H, 2.51; N, 2.91; S, 6.66. Found: C, 42.70; H, 2.55; N, 2.84; S, 6.61.

### Example 3

### (R)-2-(3-{3-[[2-Chloro-3-(trifluoromethyl)benzyl](2,2-diphenylethyl)amino]-1-methyl-propoxy}-phenyl)acetic Acid Methyl Ester

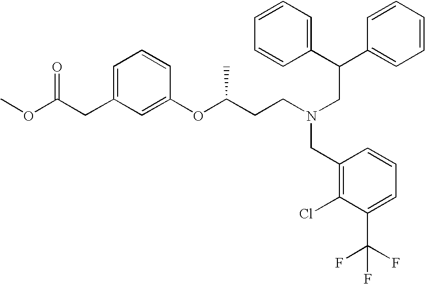 a) Toluene-4-sulfonic acid-(S)-3-hydroxy-butyl Ester

To a stirring solution of (S)-1,3-butanediol (1.0 g, 0.01 mmol) and triethylamine (1.39 g, 0.014 mmol) in dichloromethane (10 mL) at −20° C. was added dropwise p-toluenesulfonyl chloride and the mixture was stirred for 2 h. The reaction mixture was then warmed to RT and stirred overnight. The reaction mixture was poured into cold H₂O (20 mL), and extracted three times with dichloromethane. The organic extracts were then washed with brine. The organic layer was dried over sodium sulfate, filtered, and concentrated in vacuo to give 2.6 g (96% yield) of title compound as an oil. MS(ESI) 244.8(M⁺). The crude tosylate was used without further purification.

b) (S)-4-[N-(2,2-Diphenylethyl)-N-(2-chloro-3-trifluoromethyl)amino]-butan-2-ol

To a stirring solution of N-(2,2-diphenylethyl)-N-(2-chloro-3-trifluoromethyl)amine (160 mg, 0.409 mmol) and toluene-4-sulfonic acid-(S)-3-hydroxy-butyl ester (100 mg, 0.409 mmol) in acetonitrile (5 mL) was added solid K₂CO₃ (170 mg, 1.23 mmol) and NaI (184 mg 1.23 mol). The reaction mixture was heated to reflux and stirred overnight. The mixture was cooled to RT, filtered, and the filtrate was concentrated. The crude product was purified by preparative HPLC (TMC CombiPrep PDS, 75×30 mm, 25 mL/min, acetonitrile: H₂O, UV detection at 254 nm) to give 110 mg (58% yield) of the title compound as an oil. MS(ESI) 462.0(M+H⁺).

c) (R)-2-(3-{3-[[2-Chloro-3-(trifluoromethyl)benzyl](2,2-diphenylethyl)amino]-1-methyl-propoxy}-phenyl)acetic Acid Methyl Ester

To a stirring solution of (3-hydroxy-phenyl)-acetic acid methyl ester (36 mg, 0.217 mmol) in anhydrous toluene (5 mL) was added (S)₄[N-(2,2-diphenylethyl)-N-(2-chloro-3-trifluoromethyl)amino]-butan-2-ol (100 mg, 0.217 mmol). Polymer bound triphenylphosphine (115 mg, 0.346 mmol, 3 mmol/g, Fluka Chemie) was then added, and the mixture was stirred for 15 minutes. The reaction mixture was then cooled to 0° C. and diisopropylazodicarboxylate (54 mg, 0.269 mmol) was added in a dropwise fashion. The reaction mixture was stirred overnight at room temperature. The reaction mixture was next filtered and the remaining solid was washed with toluene. The filtrate was concentrated and the crude product was purified by preparative HPLC (TMC CombiPrep PDS, 75×30 mm, 25 mL/min, A: acetonitrile B: H2O, A: 85 to 100% during 10 min, UV detection at 254 nm) to give 56 mg (42% yield) of title compound as a viscous oil. MS(ESI) 610.0(M⁺).

### Example 4

### (2-Chloro-3-trifluoromethyl-benzyl)-(2,2-diphenyl-ethyl)-{3-[3-(1,2,3,4-tetrazol-5-ylmethyl)-phenoxy]-propyl}-amine

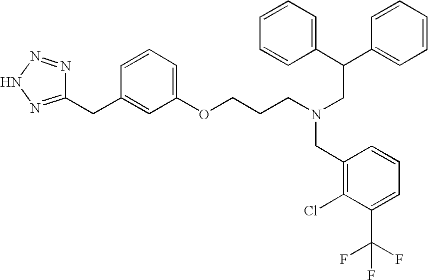

### a) 5-(3-Benzyloxy-benzyl)-1,2,3,4-tetrazole

To a stirring solution of 3-benzyloxyphenylacetonitrile (2.0 g, 8.95 mmol) in toluene (17 ml) was added trimethylsilylazide (2.37 g, 17.9 mmol) and di-n-butyltin oxide (0.22 g, 0.9 mmol). The mixture was heated at 110° C. for 48 h, and was concentrated. The reaction mixture was dissolved in ethyl acetate (100 ml) and washed two times with 10% aqueous sodium bicarbonate. The basic extracts were acidified to pH<2 with conc. HCl, and the aqueous layer was extracted with ethyl acetate. The combined organic layers were dried over sodium sulfate, filtered, and concentrated. The crude product (2.0 g, 89%) was used in the next step without further purification. MS (ESI) 267.0 (M+H⁺).

### b) 5-(3-Benzyloxy-benzyl)-ethoxymethyl-1,2,3,4-tetrazole

Mixture of Regioisomers for Ethoxymethyl Include 1- and 2-

To a stirring solution of 3-(3-benzyloxy-benzyl)-1,2,3,4-tetrazole (2.12 g, 7.96 mmol) in DMF (40 ml) at 0° C. was added NaH (0.38 g, 9.55 mmol). To this mixture was added chloromethyl ethyl ether (0.81 ml, 8.75 mmol), and the solution was stirred at RT overnight. The reaction mixture was poured into water (120 ml) and extracted three times with ethyl acetate. The ethyl acetate extracts were dried over Na₂SO₄, filtered, and concentrated. The crude mixture was subjected to column chromatography (silica gel, ethyl acetate/hexane) to provide the title compounds as a mixture of regioisomers as a light yellow oil (1.39 g, 55%). MS (ESI) 324.8 (M+).

### c) 5-(3-Hydroxy-benzyl)-ethoxymethyl-1,2,3,4-tetrazole

Mixture of Regioisomers for Ethoxymethyl Include 1- and 2-

To a stirring solution of 5-(3-benzyloxy-benzyl)-ethoxymethyl-1,2,3,4-tetrazole (mixture of regioisomers, 0.23 g, 0.71 mmol) in MeOH (5 ml) was added palladium on carbon (20 mg). The mixture was stirred for 7 h under H₂ atmosphere, filtered, and concentrated. The crude phenol was purified by preparative HPLC (TMC CombiPrep PDS, 75×30 mm, 25 mL/min, acetonitrile:H₂O, UV detection at 254 nm) to afford the desired phenol as a clear oil (0.14 g, 84%). MS (ESI) 235.0 (M+H⁺).

### d) 5-[3-(3-Bromo-propoxy)-benzyl]-(ethoxymethyl)-1,2,3,4-tetrazole

Mixture of Regioisomers for Ethoxymethyl Include 1- and 2-

A solution of 5-(3-hydroxy-benzyl)-ethoxymethyl-1,2,3,4-tetrazole (mixture of regioisomers, 132 mg, 0.56 mmol) in anhydrous toluene (5 ml) was treated with 3-bromo-propanol (117 mg, 0.84 mmol). Polymer bound triphenylphosphine (0.56 mg, 1.7 mmol, 3 mmol/g, Fluka Chemie) was then added, and the mixture stirred for 15 minutes. The reaction mixture was then cooled to 0° C. and diisopropylazodicarboxylate (166 ul, 0.84 mmol) was added dropwise. The reaction mixture was stirred at RT overnight, filtered, and the filtrate was concentrated in vacuo to give 200 mg (100% yield) of a 1:1 mixture of the title compounds as a yellow oil. MS (ES) 356.8 (M+2H⁺).

e) (2-Chloro-3-trifluoromethyl-benzyl)-(2,2-diphenyl-ethyl)-{3-[3-(ethoxymethyl-1,2,3,4-tetrazol-5-ylmethyl)-phenoxy]-propyl}-amine

Mixture of Regioisomers for Ethoxymethyl Include 1- and 2-

A solution of (2-chloro-3-trifluoromethyl-benzyl)-(2,2-diphenyl-ethyl)-{3-[3-(ethoxymethyl-1,2,3,4-tetrazol-5-ylmethyl)-phenoxy]-propyl}-amine (mixture of regioisomers, 0.2 g, 0.56 mmol) and (2,2-diphenylethyl)-(2-chloro-3-trifluoromethyl)amine (0.43 g, 1.12 mmol) in acetonitrile (10 ml) was treated with solid potassium carbonate (0.23 g, 1.7 mmol) and NaI (0.25 g, 1.7 mmol). The reaction was heated at reflux and stirred overnight. The mixture was cooled to RT, filtered, and concentrated. The crude product was purified by preparative HPLC (TMC CombiPrep PDS, 75×30 mm, 25 mL/min, acetonitrile: water, WV detection at 254 nm) to give 125 mg (33% yield) of the title compound as a viscous oil. MS (ESI) 664.2 (M⁺).

f) (2-Chloro-3-trifluoromethyl-benzyl)-(2,2-diphenyl-ethyl)-(3-[3-(1,2,3,4-tetrazol-5-ylmethyl)-phenoxy]-propyl)-amine

To a stirring solution of (2-chloro-3-trifluoromethyl-benzyl)-(2,2-diphenyl-ethyl)-{3-[3-(ethoxymethyl-1,2,3,4-tetrazol-5-ylmethyl)-phenoxy]-propyl}-amine (mixture of regioisomers, 125 mg, 0.19 mmol) in dichloromethane (11 ml) was added triethylsilane (116 mg, 1.08 mmol). The reaction mixture was treated with TFA (3 ml) and then stirred overnight. Solvent was removed and the residue was purified by preparative HPLC (TMC CombiPrep PDS, 75×30 mm, 25 mL/min, acetonitrile:water, UV detection at 254 nm) to afford 50 mg (44%) the title compound as a yellow oil. MS (ESI) 607.0(M+H⁺).

### Example 5

### (S)-2-(3-{3-[[2-Chloro-3-(trifluoromethyl)benzyl](2-phenyl-propyl)amino]propoxy}-phenyl)-acetamide

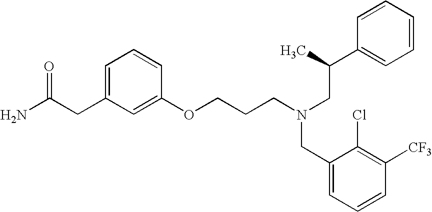 a) (S)-(2-Chloro-3-trifluoromethyl-benzyl)-(2-phenyl-propyl)-amine

To a solution of (S)-2-phenyl propylamine (0.5 g, 3.7 mmol) in dry dichloromethane was added acetic acid followed by 2-chloro-3-trifluoromethylbenzaldehyde (1.1 g, 5.5 mmol) and sodium triacetoxyborohydride (1.5 g, 7.4 mmol). After the resulting mixture was stirred for 1.5 h at RT water was added to quench the reaction. The aqueous layer was extracted with ethyl acetate. The combined organic layers were washed with brine, dried over sodium sulfate, and concentrated in vacuo. The crude mixture was purified by column chromatograph (Ethyl acetate:Hexane/25:75) to give the title compound as an oil (0.55 g, 45%). MS (ESI) 327.6 (M+H)⁺.

b) (S)-(3-{3-[[2-Chloro-3-(trifluoromethyl)benzyl](2-phenyl-propyl)amino]propoxy}-phenyl)acetic Acid Methyl Ester

A solution of (3-{3-bromo-propoxy}-phenyl)acetic acid methyl ester (0.55 g, 1.5 mmol) and (S)-(2-Chloro-3-trifluoromethyl-benzyl)(2-phenyl-propyl)-amine (0.55 g, 1.6 mmol) in acetonitrile (10 ml) was treated with solid potassium carbonate (0.4 g, 2.4 mmol). The reaction was heated to reflux and stirred for 48 h. Upon cooling to RT, the reaction was filtered through a pad of celite, washed with ethyl acetate, and the filtrate was concentrated in vacuo. The crude product was purified by column chromatograph (Ethyl acetate:Hexane/20:80) to give the title compound as an oil (0.6 g, 67%). MS (ESI) 534.6 (M+H)⁺.

c) (S)-2-(3-{3-[[2-Chloro-3-(trifluoromethyl)benzyl](2-phenyl-propyl)amino]propoxy}-phenyl)acetic Acid

A solution of (S)-(3-{3-[[2-chloro-3-(trifluoromethyl)benzyl](2-phenyl-propyl)amino]propoxy}-phenyl)acetic acid methyl ester (0.6 g, 1.1 mmol) in THF (9 ml) and water (6 ml) was treated with aqueous LiOH (1.0 N, 1.0 ml, 1.0 mmol). After stirring at RT for 2 h, additional LiOH (11.0 ml, 1.0 mmol) was added and stirring was continued for 2 h. The reaction was neutralized with acetic acid and poured into water and ethyl acetate. The layers were separated and the aqueous layer was extracted with ethyl acetate. The combined organic layers were washed with brine, dried over sodium sulfate and concentrated in vacuo. The crude mixture was purified by HPLC to give the title compound as an oil (0.4 g, 75%). MS (ESI) 520.2 (M+H)⁺.

d) (S)-2-(3-{3-[[2-Chloro-3-(trifluoromethyl)benzyl](2-phenyl-propyl)amino]propoxy}-phenyl)acetic Acid Hydrochloride Salt

To a solution of the (S)-2-(3-{3-[[2-chloro-3-(trifluoromethyl)benzyl](2-phenyl-propyl)amino]propoxy}-phenyl)acetic acid in ethyl ether was added HCl in diethyl ether (1.0M). The suspension was filtered and dried to give the title compound as a white solid (99%). NMR(400 MHz, CD₃OD) δ: 8.0 (d, J=4.0 Hz, 1H), 7.9 (d, J=4.0 Hz, 1H), 7.7-7.3 (m, 7H), 7.1 (d, J=8.0 Hz, 1H), 6.8 (m, 2H), 4.1-3.4 (m, 11H), 2.3 (m, 2H), 1.5 (d, J=4.0 Hz, 3H).

(e) (S)-2-(3-{3-[[2-Chloro-3-(trifluoromethyl)benzyl](2-phenyl-propyl)amino]propoxy}-phenyl)-acetamide

To a solution (S)-2-(3-{3-[[2-chloro-3-(trifluoromethyl)benzyl](2-phenyl-propyl)amino]propoxy}-phenyl)acetic acid hydrochloride salt (50 mg, 0.1 mmol) in dichloromethane, 1,2-dichloroethane (EDC, 19.2 mg, 0.1 mmol), 1-hydroxybenzotriazole hydrate (HOBT, 13.6 mg, 0.1 mmol), triethylamine (Et₃N, 14 μl, 0.1 mmol) and ammonia (1.0M in dioxane, 0.24 ml) were added. After the resulting mixture was stirred at room temperature for over night it was washed with 0.1N HCl, saturated NaHCO₃, water and brine. The organic layer was dried over Na₂SO₄ and concentrated under vacuum. The residue was purified with HPLC to give the tittle compound as a light yellow oil 30 mg, yield 60%. MS m/e 519.0 (M+H)⁺.

### Example 6

### 2-{2-[[2-Chloro-3-(trifluoromethyl)benzyl](2,2-diphenylethyl)amino]

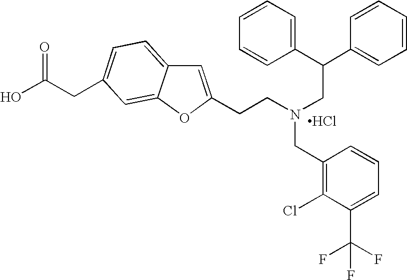 ethyl}-6-benzofuran Acetic Acid Hydrochloride F
a) (3-hydroxy-4-iodo-phenyl)-acetic Acid Methyl Ester

To a stirring solution of (3-hydroxy-phenyl)-acetic acid (5.0 g, 0.033 mole) in aqueous NH₂OH (100 mL NH₂OH (aqueous) and 50 mL H₂O) at 0° C. was added solid KI (7.6 g, 0.36 mole) and solid 12 (6.0 g, 0.030 mole). The reaction mixture was stirred for 2 h, and then poured into H₂O. The aqueous mixture was extracted three times with Et₂O, and the organic extracts were combined. The ether extracts were dried over Na₂SO₄, filtered, and concentrated. The crude product was dissolved in MeOH (100 mL), conc. HCl (2 mL) was added, and the mixture was heated at reflux overnight. The reaction was cooled to RT and concentrated. The crude methyl ester was dissolved in EtOAc, and washed two times with H₂O (50 mL). The EtOAc layer was dried over Na₂SO₄, filtered, and concentrated. The crude product was purified by preparative HPLC (TMC CombiPrep PDS, 75×30 mm, 25 mL/min, acetonitrile: H₂O, UV detection at 254 nm) to give 2.67 g (29% yield) of title compound as a white solid. MS(ESI) 292.8 (M⁺).

### b) 2-(2-hydroxy-ethyl)-6-benzofuran Acetic Acid Methyl Ester

To a stirring solution of (3-hydroxy-4-iodo-phenyl)-acetic acid methyl ester (1.04 g, 0.0035 mole) and 3-butyn-1-ol (0.5 g, 0.007 mole) in a 3:1 solution of toluene/Et₃N (25 mL) was added PPh₃ (70 mg, 0.26 mmol), CuI (68 mg, 0.35 mmol), and Pd(PPh₃)₂Cl₂ (50 mg, 0.07 mmol). The mixture was heated at 118° C. for 1 h and then cooled to RT. To the reaction mixture was added florisil (2 g) and the mixture was filtered through a fritted funnel. The crude benzofuran was concentrated and subjected to column chromatography over silica gel (silica gel 60, EM Science) using 40% EtOAc:hexane as eluent to afford 0.59 g (71% yield) of the title compound as an oil. MS (ESI) 235.0 (M+H⁺).

### c) 2-{2-[(2,2-diphenylethyl)amino]-ethyl}-6-benzofuran Acetic Acid Methyl Ester

To a stirring solution 2-[2-(2-hydroxy-ethyl)-benzofuran]acetic acid methyl ester (0.33 g, 0.0014 mole) in CH₂Cl₂ (15 mL) at 0° C. was added Et₃N (0.21 mL, 0.0015 mole) and methanesulfonyl chloride (0.12 mL, 0.0015 mole). The reaction mixture was stirred for 3 h at 0° C. The mixture was then poured into cold H₂O, and extracted two times with CH₂Cl₂ (30 mL). The CH₂Cl₂ extracts were washed with saturated aqueous NaCl, dried over Na₂SO₄, filtered, and concentrated in vacuo. The crude mesylate (prepared above) was dissolved in CH₃CN (25 mL), and the following reagents were added to the solution: solid K₂CO₃ (194 mg, 1.41 mmol) and N-2,2-diphenylethylamine (0.55 g, 0.0014 mole). The reaction mixture was heated overnight at 88° C. The mixture was filtered through a fritted funnel and concentrated. The crude product was purified by preparative HPLC (TMC CombiPrep PDS, 75×30 mm, 25 mL/min, acetonitrile: H₂O, UV detection at 254 nm) to give 125 mg (15% yield) of the title compound as a viscous oil. MS(ESI) 400.0 (M+H⁺).

### d) 2-{2-[[2-Chloro-3-(trifluoromethyl)benzyl](2,2-diphenylethyl)amino]ethyl}-6-benzofuran Acetic Acid Methyl Ester

To a stirring solution of 2-{2-[(2,2-diphenylethyl)amino]-ethyl}-6-benzofuran acetic acid methyl ester (160 mg, 0.39 mmol) and 2-chloro-3-trifluoromethylbenzaldehyde (81 mg, 0.39 mmol) in CH₂Cl₂ (4 mL) was added sodium triacetoxyborohydride (91 mg, 0.43 mmol) and two drops of glacial acetic acid. The mixture was stirred for 4 h, and was diluted with EtOAc (10 mL). The mixture was washed with saturated aqueous NH₄Cl, saturated aqueous NaHCO₃, and saturated aqueous NaCl. The organic layer was dried over Na₂SO₄, filtered, and concentrated. The crude product was purified by column chromatography over silica (Silica gel 60, EM Science) using 10% EtOAc:Hexane as eluent to afford 0.15 g (64%) of the title compound as an oil. MS(ESI) 606.2 (M⁺).

### e) 2-{2-[[2-Chloro-3-(trifluoromethyl)benzyl](2,2-diphenylethyl)amino]-ethyl}-6-benzofuran Acetic Acid Hydrochloride

To a stirring solution of 2-{2-[[2-chloro-3-(trifluoromethyl)benzyl](2,2-diphenylethyl)amino]-ethyl}-6-benzofuran acetic acid methyl ester (150 mg, 0.25 mmol) in a 4:1H₂O/THF (3 mL) solution at 0° C. was added LiOH—H₂O (23 mg, 0.55 mmol). The reaction mixture was warmed to RT and stirred overnight. The reaction mixture was concentrated to remove the THF and was diluted with H₂O (5 mL). The aqueous solution was acidified with 1 N HCl (10 mL) and extracted three times with EtOAc. The EtOAc extracts were dried over Na₂SO₄, filtered, and concentrated. The resulting tertiary amine was dissolved in Et₂O and acidified with 1 N HCl in Et₂O. The solution was stirred for 20 min. and then concentrated to afford 122 mg (78% yield) of the title compound as a white solid. MS(ESI) 592.0. (M⁺).

### Example 7

### LXR Alpha mRNA Levels are Elevated Following Transient Middle Cerebral Artery Occulsion (tMCAO) in the Rat

tMCAO: Transient (90 min) focal cerebral ischaemia was induced in male Sprague Dawley rats, each weighing between 300-350 g. The animals were initially anaesthetised with a mixture of 5% halothane, 60% nitrous oxide and 30% oxygen, placed on a facemask and anaesthesia subsequently maintained at 1.5% halothane. Middle cerebral artery occlusion (MCAO) was carried out using the intraluminal thread technique as described previously (Zea Longa, et. al., 1989). Parallel groups (n=3 per group) of animals were received either MCA occlusion or sham surgery, in which an identical procedure was followed but without insertion of the filament Animals were maintained normothermic throughout the surgical procedure, allowed to recover for 1 h in an incubator, before being singly housed. Only those animals with a neurological score of 3 1 h post-occlusion were included in the study (as assessed using a 5-point scoring system: 0, no deficit; 1, contralateral reflex; 2, weakened grip; 3, circling; 4, immobile; 5, dead). Animals were maintained for up to 4 weeks.

SYBRman quantitative PCR: The left (lesioned) cerebral cortex was dissected from each rat. All tissues were snap frozen in liquid nitrogen immediately after dissection and stored at −80° C. Tissue samples from each group were homogenised in TRIzol reagent (Life Technologies Inc., Gaithersburg, Md., USA) using 1 ml of TRIzol per 50 mg of tissue. Total RNA was extracted from the tissue according to the manufacturer's suggested protocol with the addition of an extra chloroform extraction step and phase separation, and an extra wash of the isolated RNA in 70% ethanol. The RNA was resuspended in PCR grade water and the concentration calculated by A₂₆₀ measurement. RNA quality was assessed by electrophoresis on a 1% agarose gel. Equal quantities of RNA from each animal in a group were pooled. First strand cDNA was synthesised from 1 μg of each RNA sample; 0.01M DTT, 0.5 mM each dNTP, 0.5 μg oligo(dT) primer, 40 U RNAseOUT ribonuclease inhibitor (Life Technologies Inc.), 200 U SuperscriptII reverse transcriptase (Life Technologies Inc.). Triplicate reverse transcription reactions were performed along with an additional reaction in which the reverse transcriptase enzyme was omitted to allow for assessment of genomic DNA contamination in each sample. The resulting cDNA products were divided into twenty aliquots using a Hydra 96 robot (Robbins Scientific, Sunnyvale, Calif., USA) for parallel SYBRman PCR reactions using different primer sets for quantification of multiple cDNA sequences. SYBRman PCR was carried out using an ABI prism 7700 sequence detector (Applied Biosystems, Foster City, Calif., USA) on the cDNA samples; using SYBRgreen PCR Master Mix (Applied Biosystems) 50° C. for 2 minutes, 95° C. for 10 minutes followed by forty cycles of 95° C. for 15 seconds, 60° C. for 1 minute. Additional reactions were performed on each 96 well plate using known dilutions of rat genomic DNA (Clontech Laboratories Inc., Palo Alto, Calif., USA) as a PCR template to allow construction of a standard curve relating threshold cycle to initial template copy number.

Primer sequences were as follows:

| | |
|---|---|
| LXR-alpha left primer: | AGTGTTTGCACTTCGCCTGC |
| LXR-alpha right primer: | GTAAGCTTCAGCTGCGTGGC |

### Example 8

### LXR Agonists Promote Neurite Outgrowth

Hippocampal neurons: The hippocampi of gestational day 18 rat embryos were dissected out, incubated in trypsin (0.08%, 30 min at 37° C.) and dissociated mechanically (16). Hippocampal cells were resuspended in neurobasal medium supplemented with B27, anti-oxidants, 1 mM glutamine, 25 μM glutamate, 1 mM pyruvate. For outgrowth assays, cells were plated at a density of 3000 cells/well into 96 well dishes that had previously been coated with poly-D-lysine followed by 10% FCS and cultured for 48 hours.

Cortical neurons: Cortex from gestational day 18-20 rat embryos were collected in HBSS on ice. Cells were dissociated as described for hippocampal neurons. Cells were pelted (200 g, 5 mins and resuspended in medium as described for hippocampal cells. Cells were plated at 6000 cells/well and cultured for 24 hours.

The test compound was solubilised in DMSO and added to culture medium at time of cell plating at a dilution of 1:1000. Vehicle only (1:1000) was added to culture medium of untreated controls. Cells were fixed with 4% paraformaldehyde for 1 hour on ice, washed with PBS and stained using Coomassie. Assays were quantified using a KS300 image analysis system (Imaging Associates, UK). For each cell measured, the length from the edge of the cell to the end of the longest neurite was measured for 100 cells/well for each treatment in triplicate. All data are means and SEM pooled from three independent experiments. Results are expressed as a percentage of the length of neurites of cells treated with vehicle alone.

| Concentration of test | HC neurons | | CR neurons |
|---|---|---|---|
| compound (μm) | Example 1 | 22(R) hydroxychol. | Example 1 |
| 0 | 100 (6.2) | 100 (5.4) | 100 (1) |
| 0.3 | 109 (6.25) | 115.9 (6.02) | 105 (1.79) |
| 1.0 | 113 (5.05) | 116 (7.45) | 110 (2.4) |
| 3.0 | 120 (7.75) | 129.9 (4.12) | 123.6 (6.6) |
| 10.0 | 147 (11.8) | 127.9 (6.18) | 126.23 (2.5) |

Figures in parentheses represent the standard error from pooled data from three independent experiments (HC) or from triplicate wells in a single experiment (CR).

### Example 9

### LXR Agonists are Anti-Neuroinflammatory

NTW8 mouse microglial cells were plated into a 96 well plate at a density of 2×10⁵ cells/well in DMEM supplemented with 10% FCS, 2 mM glutamine, 10 ng/ml basic fibroblast growth factor (R&D Systems) and N-2 (Gibco). Next day, cell were stimulated for 24 hrs in DMEM containing 10 ng/ml LPS (Sigma) and 20 U/ml IFN-γ (Gibco) in the presence of increasing concentrations of the test compound solubilised in DMSO. Media was removed after 24 hrs and analysed by ELISA for secreted IL-6, TNF-α (R&D systems) and PGE2 (Amersham) or via a Greiss assay for nitric oxide (NO) production. Cell viability was assessed by an MTT assay (Promega). All data are means and SEM pooled from two independent experiments. Results are expressed as a percentage of the LPS\INF-γ stimulated control cells treated with DMSO alone.

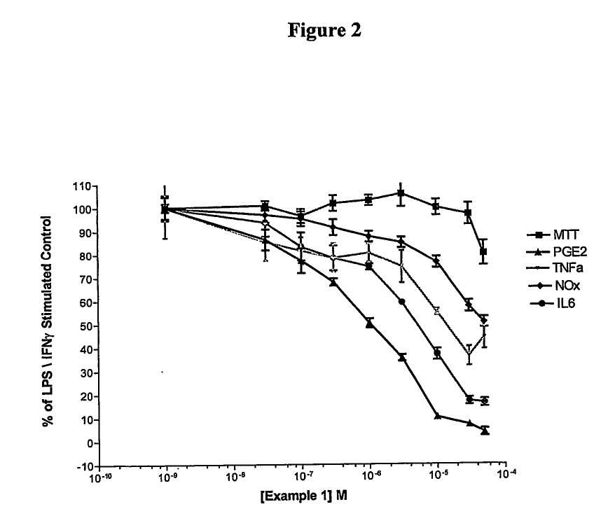

FIG. 2 shows that Example 1 inhibited the secretion of pro-inflammatory mediators (IL-6, PGE2, TNF-α and NO) from LPS INF-γ stimulated microglia cells.

### Example 10

### LXR Agonists Promote Astroglial Cell Cholesterol Efflux

It has recently been reported that astroglial cells increase synapse plasticity by secreting cholesterol-rich lipoprotein particles (22). These particles are internalized by neurons, leading to an increase in the number and efficacy of synapses. Therefore it is possible that compounds which stimulate astroglial cell cholesterol efflux would promote synaptogenesis, and thus aid nerve regeneration.

Primary murine neuronal cultures were prepared from C57 B1/6 mice essentially as described elsewhere (23). In brief, embryonic day 18 fetuses were collected by caesarian section, their brains removed and the cerebral cortices dissected from the rest of the brain. The tissue was rinsed during these steps several times in Ca²⁺- and Mg²⁺-free Hank's balanced salt solution (HBSS, containing 1 mM HEPES, GIBCO). After the meninges were removed with forceps, the tissue was minced and incubated for 15 minutes at 37° C. in 0.25% trypsin (Sigma) in HBSS. The tissue was then washed twice in HBSS and twice in neuronal plating media (minimal essential media [MEM] containing 3 mg/ml glucose, 5% fetal bovine serum [FBS; GibcoBRL], 5% horse serum [HS; GibcoBRL], 100 U/ml penicillin/100 μg/ml streptomycin [Irvine Scientific] and 2 mM glutamine [Irvine Scientific]) to which 10 μg/ml DNAseI (Sigma) had been added. The tissue was then triturated and spun at 3000×g for 10 minutes. The resulting cell pellet was resuspended in plating media, and trypan blue-excluding surviving cells were counted in a hemacytometer. Cells were plated into 6-well plates at 1.35×10⁶ cells per well and maintained at 37° C. in 5% CO₂/95% air in a humidified incubator. The next morning, the plating media from some cultures was carefully withdrawn and replaced with serum-free media (Neurobasal media containing B27 supplement [both from GibcoBRL], 100 U/ml penicillin/100 μg/ml streptomycin, and 0.5 mM glutamine). These cells were fed by half-volume exchange with fresh serum-free media on day 3 in culture. Serum-free growth conditions restrict glial outgrowth such that the resulting cultures are >95% neuronal. The remaining cells were maintained in the same serum-containing plating media without media exchange to establish cultures composed of neurons and glia in an approximate ratio of 60:40 (23). Cells were used in cholesterol efflux assays starting on culture day 6.

Murine astroglia were obtained from postnatal day 1 pups. Briefly, pups were decapitated, their brains removed, and the cerebral cortices prepared as described previously (45), except that astrocyte plating media was used (Dulbecco's modified eagle media [DMEM] containing 4 mg/ml glucose, 5% FBS and 5% HS [GibcoBRL or Irvine Scientific], 100 U/ml penecillin/100 μg/ml streptomycin, 25 mM HEPES, and 2-4 mM glutamine). Glia were grown in T75 flasks at a density of approximately 2 brains per flask. Cells were fed once weekly by complete media exchange in maintenance media (DMEM containing 4.5 mg/ml glucose, 10% FBS, 100 U/ml penecillin/100 μg/ml streptomycin, 25 mM HEPES, and 6 mM glutamine). By visual inspection, these cultures were nearly entirely astroglial with <1% contamination with microglia. After 7-14 days in vitro, cells were collected by trypsinization, counted in a hemacytometer, and plated into 6-well plates at 50-100,000 cells per well in maintenance media. Cholesterol efflux assays were begun after 3 days' growth, by which time the cells were approximately 40% confluent.

Cholesterol efflux assays were performed as described elsewhere (24) with some modifications. For astrocytes, the culture media was removed and replaced with 1 ml/well DMEM containing 4.5 mg/ml glucose, 5% FBS, 100 U/ml penecillin/100 μg/ml streptomycin, 25 mM HEPES, and 6 mM glutamine supplemented with 0.5% BSA and 5 W [1,2-³H(N)]-cholesterol (1 mCi/ml ethanolic stock). Twenty-four hours later, cells were washed once in serum-free DMEM containing glucose, penicillin/streptomycin, HEPES, and glutamine and then incubated for 24 hours in the same media supplemented with 0.5% BSA and various drugs or DMSO vehicle. The next day, cells were washed twice in serum-free media and then incubated for a further 24 hours in 1 ml/well of serum-free media supplemented with drugs or DMSO. Human ApoA-1 was added to some cultures to serve as an exogenous cholesterol acceptor molecule. At the end of this incubation, culture media was collected and spun in a microfuge. Adherent cells were washed three times in PBS and extracted for 1 hr in 1 ml per well hexane:isopropanol (3:2 vol:vol). Two hundred microliters of the culture media supernatant and 200 μl of the cell extract were counted for tritium in 2 ml Packard Ultima Gold Scint. Cholesterol efflux from neurons was examined in much the same way except that cells were always washed and incubated with the neuronal serum-free culturing media described above. On the first day of the efflux experiment, neurons received a half-volume media change with media containing 10 μl [1,2-³H(N)]-cholesterol. Efflux is expressed as percent of the total radiolabeled cholesterol pool present in the cultures.

Cholesterol efflux as a percentage of total
[³H] cholesterol

| Compound | Astrocytes | Neurons |
|---|---|---|
| Vehicle | 1.835 (0.826) | 1.256 (0.329) |
| Example 1 | 4.722 (0.783)* | 1.252 (0.179) |
| T0901317 | 4.370 (0.561)* | 1.427 (0.031) |

Basal efflux from astrocyte cultures was enhanced by each LXR agonists. Data reflect 3 independent determinations and are representative of 4 separate experiments. Figures in parentheses represent the standard deviation. *, p<0.05 by t-test relative. LXR agonists promote cholesterol efflux from primary astroglial cells, but not neurons.

### Example 11

### LXR Agonists Upregulate Target Gene Expression in Murine Primary Astrocyte and Neuron Cell Cultures

Total RNA was isolated from tissue and cell culture samples using TRIzol reagent (GibcoBRL). Briefly, tissue samples were thawed directly in 1 ml Trizol/50 mg tissue and homogenized with a polytron. To facilitate recovery of nucleic acid, 100 μg glycogen (Ambion) was added. Cultured cells were lysed in 1 ml Trizol containing 100 μg glycogen per well. Samples were then extracted in chloroform and spun at 4° C. at 11,000×g for 15 min. The aqueous phase was collected and the RNA was precipitated with the addition of isopropanol. The samples were then spun at 11,000×g at 4° C. for 15 min, the pellet washed in 75% ethanol, dried, and dissolved in water. Samples were stored at −70° C. until use.

Total RNA samples were diluted to 100 μg/ml and treated with 40 units/ml RNA-free DNAse-I (Ambion) for 30 min at 37° C. followed by inactivation at 75° C. for 5 min. Samples were quantitated by spectrophotometry or with the RiboGreen assay (Molecular Probes) and diluted to a concentration of 10 ng/μl. Samples were then assayed in duplicate or triplicate 25-μl reactions using 25 ng RNA per reaction with Perkin Elmer chemistry on an ABI Prism 7700 (Perkin Elmer) according to manufacturer's instructions. Gene-specific primers were used at 7.5 or 22.5 pmol per reaction, optimized for each gene examined, and the gene-specific probe was used at 5 pmol per reaction. Primers and probe were synthesized by Keystone Labs (Camarillo, Calif.). In this system, the probe is degraded by Taq polymerase during the amplification phase, releasing the fluorescent tag from its quenched state; amplification data is expressed as the number of PCR cycles required to elevate the fluorescence signal beyond a threshold intensity level. Fold induction values were calculated by subtracting the mean threshold cycle number (Ct) for each treatment group from the mean Ct for the vehicle group and raising 2 to the power of this difference.

Total RNA prepared from sister cultures treated in parallel was used to profile the expression of ABCA1, ABCG1, and SREBP1c. Expression levels for each gene in neurons and astrocytes were normalized to the vehicle-treated group and are from 2-3 separate experiments. Target gene expression was more highly induced by drug treatment in astrocyte cultures than neuronal cultures.

| | Compound | | | | | |
|---|---|---|---|---|---|---|
| | ABCA1 | | | | | |
| | Astro- | | ABCG1 | | SREBP-1c | |
| | cytes | Neurons | Astrocytes | Neurons | Astrocytes | Neurons |
| Vehicle | 1.0 | 1.0 | 1.0 | 1.0 | 1.0 | 1.0 |
| Example 1 | 11.8 | 2.8 | 14.9 | 2.8 | 13.9 | 3.1 |
| T0901317 | 18.4 | 3.0 | 12.5 | 3.1 | 20.2 | 3.1 |

### Example 12

### LXR Agonists Upregulate Target Gene Expression in the CNS

Adult male C57 B1/6 mice (3 per group) were dosed by oral gavage with the LXR agonists Example 1, T0901317, or vehicle (0.5% methylcellulose). Example 1 was delivered at 10 mg kg twice daily, while T0901317 was administered at 50 mg/kg once daily. After 3 or 7 days' treatment, animals were killed and their brains removed. The cerebellum and both hippocampi were dissected and snap frozen in liquid nitrogen for RNA isolation. Total RNA was prepared from the hippocampus and cerebellum (black bars) and analyzed for gene expression patterns using QRT-PCR (as described above). Expression levels for each gene in each tissue were normalized to the average expression level in the vehicle group. Both LXR agonists enhanced ABCA1 expression relative to the vehicle-treated group, with the effect most pronounced in the cerebellum after 3 days' treatment.

| | Compound | | | | | |
|---|---|---|---|---|---|---|
| | ABCA1 | | ABCG1 | | SREBP-1c | |
| | HC | CB | HC | CB | HC | CB |
| Vehicle | 1.0 (0.092) | 1.0 (0.049) | 1.0 (0.112) | 1.0 (0.153) | 1.0 (0.060) | 1.0 (0.076) |
| Example 1 | 1.57 (0.348) | 2.13 (0.354) | 1.14 (0.212) | 1.52 (0.073) | 2.22 (0.600) | 2.05 (0.427) |
| T0901317 | 2.70 (0.304) | 3.66 (0.389) | 1.22 (0.098) | 2.31 (0.081) | 2.68 (0.209) | 3.44 (0.632) |

| | Compound | | | | | |
|---|---|---|---|---|---|---|
| | ABCA1 | | ABCG1 | | SREBP-1c | |
| | HC | CB | HC | CB | HC | CB |
| Vehicle | 1.0 (0.122) | 1.0 (0.044) | 1.0 (0.072) | 1.0 (0.060) | 1.0 (0.096) | 1.0 (0.174) |
| Example 1 | 1.26 (0.162) | 1.43 (0.302) | 0.89 (0.045) | 1.17 (0.142) | 0.71 (0.327) | 1.75 (0.236) |
| T0901317 | 2.69 (0.175) | 2.94 (0.084) | 1.37 (0.092) | 1.80 (0.116) | 2.16 (0.110) | 2.97 (0.557) |

### REFERENCES

1. Fawcett & Asher, 1999. The glial scar and central nervous system repair. Brain Res. Bulletin, 49 : 377-391
2. Horner & Gage. Regenerationg the damaged central nervous system. 2000, Nature 407 963-970
3. McGeer P L, McGeer E G. (1995) The inflammatory response system of brain: implications for therapy of Alzheimer and other neurodegenerative diseases. Brain Res Brain Res Rev. 21(2):195-218
4. Peet D J, Janowski B A, Mangelsdorf D J. The LXRs: a new class of oxysterol receptors. Curr Opin Genet Dev 1998;8(5):571-5.
5. Apfel R, Benbrook D, Lernhardt E, Ortiz M A, Salbert G, Pfahl M. A novel orphan receptor specific for a subset of thyroid hormone-responsive elements and its interaction with the retinoid/thyroid hormone receptor subfamily. Mol Cell Biol 1994; 14(10):7025-35.
6. Teboul M, Enmark E, Li Q, Wikstrom A C, Pelto-Huikko M, Gustafsson J A. OR-1, a member of the nuclear receptor superfamily that interacts with the 9-cis-retinoic acid receptor. Proc Natl Acad Sci USA 1995;92(6):2096-100.
7. Song C, Kokontis J M, Hiipakka R A, Liao S. Ubiquitous receptor: a receptor that modulates gene activation by retinoic acid and thyroid hormone receptors. Proc Natl Acad Sci USA 1994;91(23):10809-13.
8. Willy P J, Umesono K, Ong E S, Evans R M, Heyman R A, Mangelsdorf D J. LXR, a nuclear receptor that defines a distinct retinoid response pathway. Genes Dev 1995; 9(9):1033-45.
9. Peet D J, Turley S D, Ma W, Janowski B A, Lobaccaro J M, Hammer R E, et al. Cholesterol and bile acid metabolism are impaired in mice lacking the nuclear oxysterol receptor LXR alpha. Cell 1998; 93(5):693-704.
10. Luo Y, Tall A R. Sterol upregulation of human CETP expression in vitro and in transgenic mice by an LXR element. J Clin Invest 2000; 105(4):513-20.22
11. Repa J J, Liang G, Ou J, Bashmakov Y, Lobaccaro J M, Shimomura I, et al. Regulation of mouse sterol regulatory element-binding protein-1c gene (SREBP-1c) by oxysterol receptors, LXRalpha and LXRbeta. Genes Dev 2000; 14(22):2819-30.
12. Schultz J R, Tu H, Luk A, Repa J J, Medina J C, Li L, et al. Role of LXRs in control of lipogenesis. Genes Dev 2000;14(22):2831-8.
13. Laffitte B A, Repa J J, Joseph S B, Wilpitz D C, Kast H R, Mangelsdorf D J, et al. LXRs control lipid-inducible expression of the apolipoprotein E gene in macrophages and adipocytes. Proc Natl Acad Sci USA 2001; 98(2):507-12.
14. Costet P, Luo Y, Wang N, Tall A R. Sterol-dependent transactivation of the ABC1 promoter by the liver X receptor/retinoid X receptor. J Biol Chem 2000; 275(36):28240-5.
15. Repa J J, Turley S D, Lobaccaro J A, Medina J, Li L, Lustig K, et al. Regulation of absorption and ABC1-mediated efflux of cholesterol by RXR heterodimers. Science 2000; 289(5484): 1524-9.
16. Venkateswaran A, Repa J J, Lobaccaro J M, Bronson A, Mangelsdorf D J, Edwards P A. Human white/murine ABC8 mRNA levels are highly induced in lipid-loaded macrophages. A transcriptional role for specific oxysterols. J Biol Chem 2000; 275(19): 14700-7.
17. Venkateswaran A, Laffitte B A, Joseph S B, Mak P A, Wilpitz D C, Edwards P A, et al. Control of cellular cholesterol efflux by the nuclear oxysterol receptor LXR alpha. Proc Natl Acad Sci USA 2000; 97(22):12097-102.
18. Schwartz K, Lawn R M, Wade D P. ABC1 gene expression and ApoA-I-mediated cholesterol efflux are regulated by LXR. Biochem Biophys Res Commun 2000;274(3): 794-802.
19. Schmidt, A., Vogel, R., Holloway, M. K., Rutledge, S. J., Friedman, O., Yang, Z., Rodan, G. A. and Friedman, E. Transcription control and neuronal differentiation by agents that activate the LXR nuclear receptor family. (1999) Mol. and Cell. Endoc., 155: 51-60.
20. Virley, D., Beech, J. S., Smart, S. C., Williams, S. C. Hodges, H & Hunter, A. J. A temporal MRI assessment of neuropathology after transient middle cerebral artery occlusion in the rat; correlations with behaviour. J. Cereb. Blood Flow Metab. 2000, 20, 563-582
21. Skaper S D, Facci L, Milani D, Leon A, and Toffano G (1990). In Methods in Neurosciences, Vol 2, Academic Press, San Diego.
22. Mauch D H, Nagler K, Schumacher S, Goritz C, Muller E C, Otto A, Pfrieger F W. (2001) CNS synaptogenesis promoted by glia-derived cholesterol. Science. 294(5545): 1354-7
23. Whitney, K. D. & McNamara, J. O. Glu R3 autoantibodies destroyy neural cells in a complement dependent manner modulated by complement regulatory proteins. Journal of Neuroscience. 2000; 20(19):7307-16
24. Oliver, W. J., Shenk, J. L., Snaith, M. R., Russell, C. S., Plunket, K. D., Bodkin, N. L., Lewis, M. C., Winegar, D. A., Sznaidman, M. L., Lambert, M. H., Xu H. E., Sternbach, D. D., Kliewer, S. A., Hansen, B. C., and Willson, T. M. A selective peroxisome proliferator-activated receptor agonist promotes reverse cholesterol transport (2001) Proc Natl Acad Sci USA 98(9), 5306-5311.
25. Repa J J, Mangelsdrof D J 2000 The role of orphan nuclear receptors in the regulation of cholesterol homeostasis. Annu Rev Cell Dev Biol 16:459-481.
26. Teboul M, Enmark E, Li Q, Wikstrom A C, Pelto-Huikko M, Gustafsson J A 1995 OR-1, a member of the nuclear receptor superfamily that interacts with the 9-cis-retinoic acid receptor. Proc Natl Acad Sci USA 92:2096-2100.
27. Janowski B A, Willy P J, Devi T R, Falck J R, Mangelsdorf D J 1996 An oxysterol signalling pathway mediated by the nuclear receptor LSR alpha. Nature 383:728-731.
28. Lehman J M, Kliewer S A, Moore L B et al. 1997 Activation of the nuclear receptor LXR by oxysterols defines a new hormone response pathway. J. Biol. Chem. 272:3137-3140.
29. Dietschy J M, Turley S D 2001 Cholesterol metabolism in the brain. Curr Opin Lipidol 12:105-112.
30. Lutjohann, D, Breuer O, Ahlborg G et al. 1996 Cholesterol homeostasis in human brain: evidence for an age-dependent flux of 24S-hydroxycholesterol from the brain into the circulation. Proc Natl Acad Sci USA 93: 9799-9804.
31. Bjorkhem I, Lutjohann D, Breuer O, Sakinis A, Wennmalm A 1997 Importance of a novel oxidative mechanism for elimination of brain cholesterol. Turnover of cholesterol and 24(S)-hydroxycholesterol in rat brain as measured with 1802 techniques in vivo and in vitro. J Biol Chem 272: 30178-30184.
32. Bjorkhem I, Lutjohann D, Diczfalusy U, Stahle L, Ahlborg G, Wahren J 1998 Cholesterol homeostasis in human brain: turnover of 24S-hydroxycholesterol and evidence for a cerebral origin of most of the oxysterol in the circulation. J. Lipid Res. 39: 1594-1600.
33. Xie C, Burns D K, Turley S D, Dietschy J M 2000 Cholesterol is sequestered in the brains of mice with Niemann-Pick type C disease but turnover is increased. J Neuropathol Exp Neurol 59: 1106-1117.
34. Lutjohann D, Papassotiropoulos A, Bjorkhem I et al. 2000 Plasma 24S-hydroxycholesterol (cerebrosterol) is increased in Alzheimer and vascular demented patients. J Lipid Res 41: 195-198.
35. Notkola I L, Sulkava R, Pekkanen J et al 1998 Serum total cholesterol, apolipoprotein E epsilon 4 allele, and Alzheimer's disease. Neuroepidemiology 17: 14-20.
36. Kuo Y M, Emmerling M R, Bisgaier C L et al. 1998 Elevated low-density lipoprotein in Alzheimer's disease correlates with brain abeta 1-42 levels. Biochem Biosphys Res Commun 252: 711-715.
37. Wolozin B, Kellman W, Ruosseau P, Celesia G G, Siegel G 2000 Decreased prevalence of Alzheimer disease associated with 3-hydroxy-3-methyglutarly coenzyme A reductase inhibitors. Arch Neurol 57: 1439-1443.
38. Jick H, Zornberg G L, Jick S S, Seshardi S, Dracbman D A 2000 Statins and the risk of dementia. Lancet 356:1627-1637.
39. Strittmatter W J, Saunders A M, Schmechel D et al. 1993 Apolipoprotein E: high-avidity binding to beta-amyloid and increased frequency of type 4 allele in late-onset familial Alzheimer disease. Proc Natl Acad Sci USA 90: 1977-1981.
40. Roses A D, Saunders A M, Alberts M A et al. 1995 Apolipoprotein E E4 allele and risk of dementia. JAMA 273:374-375; discussion 375-376.
41. Strittmatter W J, Roses A D 1996 Apolipoprotein E and Alzheimer's disease. Annu Rev Neurosci 19:53-77.
42. Jarvik G P, Wijsman E M, Kukull W A, Schellenberg G D, Yu C, Larson E B 1995 Interactions of apolipoprotein E genotype, total cholesterol level, age, and sex in prediction of Alzheimer's disease: a case-control study. Neurology 45:1092-1096.
43. Liu H C, Hong C J, Wang S J et al. 1999 ApoE genotype in relation to AD and cholesterol: a study of 2,326 Chinese adults. Neurology 53:662-966.
44. Lund E G, Guileyardo J M, Russell D W 1999 cDNA cloning of cholesterol 24-hydroxylase, a mediator of cholesterol homeostasis in the brain. Proc Natl Acad Sci USA 96:7238-7243.

The above description fully discloses how to make and use the present invention. However, this invention is not limited to the particular embodiments described hereinabove, but includes all modification thereof within the scope of the appended claims and their equivalents. Those skilled in the art will recognize through routine experimentation that various changes and modifications can be made without departing from the scope of this invention. The various references to journals, patents and other patent applications that are cited herein are incorporated by reference herein as though fully set forth.

1. A method for treating a patient suffering from a disease selected from the group consisting of: stroke, Alzheimer's disease, fronto-temporal dementias, peripheral neuropathy, Parkinson's disease, dementia with Lewy bodies, Huntington's disease, amyotrophic lateral sclerosis, and multiple sclerosis, said method comprising the step of administering to said patient an effective amount of an LXR modulator in combination with a carrier.

2. The method as claimed in claim 1, wherein said LXR modulator is selected from the group consisting of: an LXR agonist and an LXR antagonist.

3. A method for promoting cholesterol efflux in at least one astroglial cell, said method comprising the step of: contacting said at least one astroglial cell with a cholesterol-efflux-promoting effective amount of an LXR modulator in combination with a carrier.

4. The method according to claim 3, wherein said LXR modulator is selected from the group consisting of: an LXR agonist and an LXR antagonist.

5. A method for treating a patient suffering from a disease or disorder characterised by neuron degeneration, inflammation in the CNS, injury or impaired plasticity, said method comprising the step of administering to said patient an effective amount of an LXR modulator in combination with a carrier.

6. The method according to claim 5, wherein said LXR modulator is selected from the group consisting of: an LXR agonist and an LXR antagonist.

7. The method according to claim 1 or, wherein the disease is selected from psychiatric disorders such as schizophrenia and depression.

8. The method according to claim 1, wherein the disease is selected from conditions associated with head or spinal cord injury, including trauma.

9. The method according to claim 1, wherein the LXR modulator comprises a compound of formula (I) 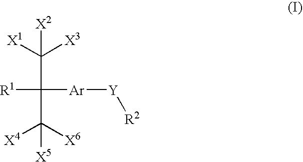
wherein:
Ar represents an aryl group; R¹ is —OH, —O—(C₁-C₇)alkyl, —OC(O)—(C₁-C₇)alkyl, —O—(C₁-C₇)heteroalkyl, —OC(O)— (C₁-C₇)heteroalkyl, —CO₂H, —NH₂, —NH(C₁-C₇)alkyl, —N((C₁-C₇)alkyl)₂ or —NH—S(O)₂—(C₁-C₅)alkyl;
R² is (C₁-C₇)alkyl, (C₁-C₇)heteroalkyl, aryl and aryl(C₁-C₇)alkyl;
X¹, X², X³, X⁴, X⁵ and X⁶ are each independently H, (C₁-C₅)alkyl, (C₁-C₅)hetroalkyl, F or Cl, with the proviso that no more than three of X¹ through X⁶ are H, (C₁-C₅)alkyl or (C₁-C₅)heteroalkyl; and
Y is —N(R¹²)S(O)ₘ—, —N(R¹²)S(O)ₘN(R¹³), —N(R¹²)C(O)—, —N(R¹²)C(O)N(R¹³), —N(R¹²)C(S)— or —N(R¹²)C(O)O—, wherein R¹² and R¹³ are each independently hydrogen, (C₁-C₇)aryl, (C₁-C₇)heteroalkyl, aryl and aryl(C₁-C₇)alkyl, and optionally when Y is —N(R¹²)S(O)ₘ— or —N(R¹²)S(O)ₘN(R¹³)—, R¹² forms a five, six or seven-membered ring fused to Ar or to R² through covalent attachment to Ar or R², respectively. In the above Y groups, the subscript m is an integer of from 1 to 2;
or a pharmaceutically acceptable derivative thereof

10. The method according to claim 1, wherein the LXR modulator comprises a compound of formula (II): 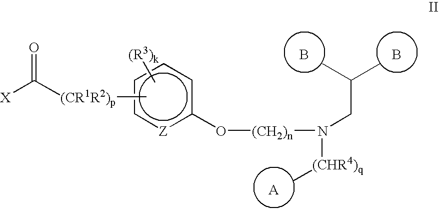
wherein:
X is OH or NH₂;
p is 0-6;
each R¹ and R² are the same or different and are each independently selected from the group consisting of H, C₁₋₈alkyl, C₁₋₈alkoxy and C₁₋₈thioalkyl;
Z is CH or N;
when Z is CH, k is 0-4;
when Z is N, k is 0-3;
each R³ is the same or different and is independently selected from the group consisting of halo, —OH, C₁₋₈alkyl, C₂₋₈alkenyl, C₁₋₈alkoxy, C₂₋₈alkenyloxy, —S(O)ₐR⁶, —NR⁷R⁸, —COR⁶, COOR⁶, R¹⁰COOR⁶, OR¹⁰COOR⁶, CONR⁷R⁸, —OC(O)R⁹, —R¹⁰NR⁷R⁸, —OR¹⁰NR⁷R⁸, 5-6 membered heterocycle, nitro, and cyano;
a is 0, 1 or 2;
R⁶ is selected from the group consisting of H, C₁₋₈alkyl, C₁₋₈alkoxy and C₂₋₈alkenyl;
each R⁷ and R⁸ are the same or different and are each independently selected from the group consisting of H, C₁₋₈alkyl, C₂₋₈alkenyl, C₃₋₈alkynyl;
R⁹ is selected from the group consisting of H, C₁₋₈alkyl and —NR⁷R⁸;
R¹⁰ is C₁₋₈alkyl;
n is 2-8;
q is 0 or 1;
R⁴ is selected from the group consisting of H, C₁₋₈alkyl, C₁₋₈alkenyl, and alkenyloxy;
Ring A is selected from the group consisting of C₃₋₈cycloalkyl, aryl, 4-8 membered heterocycle, and 5-6 membered heteroaryl;
each ring B is the same or different and is independently selected from the group consisting of C₃₋₈cycloalkyl and aryl:
or a pharmaceutically acceptable derivative thereof.

11. The method according to claim 1, wherein the LXR modulator comprises a compound of formula (III): 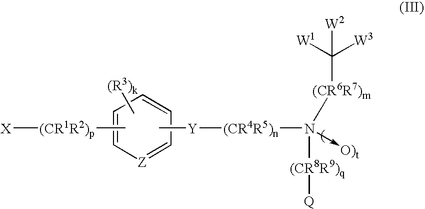
wherein:
X is selected from C₁-C₈ alkyl, halo, —OR¹⁰, —NR¹⁴R¹⁵, nitro, cyano, —COOR¹⁰, —COR¹³, —OCOR¹³, —CONR¹⁴R¹⁵, —N(R¹⁷)COR¹³, —N(R¹⁷)CONR¹⁴R¹⁵, —N(R¹⁷)COOR¹³, —SO₃H, —SO₂NR¹⁴R¹⁵, —C(═NR¹⁷)NR¹⁴R¹⁵, —N(R¹⁷)SO₂R¹⁶, and a 5 or 6-membered heterocyclic group;
or X and an adjacent R³, taken together with the atoms to which they are bonded, form an alkylenedioxy moiety;
Z is CH, CR³ or N, wherein when Z is CH or CR³, k is 0-4 and t is 0 or 1, and when Z is N, k is 0-3 and t is 0;
Y is selected from —O—, —S—, —N(R¹⁰)—, and —C(R⁴)(R⁵)—;
W¹ is selected from C₁-C₆ alkyl, C₃-C₈ cycloalkyl, aryl and Het, wherein said C₁-C₈ alkyl, C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl, is optionally unsubstituted or substituted by one or more halo substituents;
W² is selected from H, halo, C₁-C₆ alkyl, C₂-C₆ alkenyl, C₂-C₆ alkynyl, —C₀-C₆ alkyl-NR¹¹R¹², —C₀-C₆ alkyl-SR¹¹, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹² CONR¹¹R¹², —C₀-C₆ alkyl-NR¹¹COR¹³, —C₀-C₆ alkyl-Het, —C₀-C₆ alkyl-Ar and —C₀-C₆ alkyl-C₃-C₇ cycloalkyl, wherein said C₁-C₆ alkyl is optionally unsubstituted or substituted by one or more halo substituents, and wherein the C₃-C₇ cycloalkyl, Ar and Het moieties of said —C₀-C₆ alkyl-Het, —C₀-C₆ alkyl-Ar and —C₀-C₆ alkyl-C₃-C₇ cycloalkyl are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl, is optionally unsubstituted or substituted by one or more halo substituents;
W³ is selected from the group consisting of: H, halo, C₁-C₆ alkyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R^{o}, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₁-C₆ alkyl-Ar and —C₁-C₆ alkyl-C₃-C₇ cycloalkyl, wherein said C₁-C₆ alkyl is optionally unsubstituted or substituted by one or more halo substituents;
Q is selected from C₃-C₈ cycloalkyl, Ar and Het; wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹²—C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl is optionally unsubstituted or substituted by one or more halo substituents;
p is 0-8;
n is 2-8;
m is 0 or 1;
q is 0 or 1;
t is 0 or 1;
each R¹ and R² are independently selected from H, halo, C₁-C₆ alkyl, C₃-C₆ alkenyl, C₃-C₆ alkynyl, —C₀-C₆ alkyl-NR¹¹R¹², —C₀-C₆ alkyl-OR¹⁰, —C₀-C₆ alkyl-SR¹⁰, —C₁-C₆ alkyl-Het, —C₁-C₆ alkyl-Ar and —C₁-C₆ alkyl-C₃-C₇ cycloalkyl, or R¹ and R² together with the carbon to which they are attached form a 3-5 membered carbocyclic or heterocyclic ring, wherein said heterocyclic ring contains one, or more heteroatoms selected from N, O, and S, where any of said C₁-C₆ alkyl is optionally unsubstituted or substituted by one or more halo substituents;
each R³ is the same or different and is independently selected from halo, cyano, nitro, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SR₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, wherein said C₀-C₆ alkyl is optionally unsubstituted or substituted by one or more halo substituents;
each R⁴ and R⁵ is independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R⁶ and R⁷ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R⁸ and R⁹ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R¹⁰ is selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;
each R¹¹ and each R¹² are independently selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl, or R¹¹ and R¹² together with the nitrogen to which they are attached form a 4-7 membered heterocyclic ring which optionally contains one or more additional heteroatoms selected from N, O, and S;
R¹³ is selected from C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;
R¹⁴ and R¹⁵ are each independently selected from H, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-O—Ar, —C₀-C₆ alkyl-O-Het, —C₀-C₆ alkyl-O—C₃-C₇ cycloalkyl, —C₀-C₆ alkyl-S(O), —C₁-C₆ alkyl, —C₀-C₆ alkyl-S(O)ₓ—Ar, —C₀-C₆ alkyl-S(O)ₓ-Het, —C₆-C₆ alkyl-S(O), —C₃-C₇ cycloalkyl, —C₀-C₆ alkyl-NH-Het, —C₀-C₆ alkyl-NH—C₃-C₇ cycloalkyl, —C₀-C₆ alkyl-N(C₁-C₄ alkyl)-Ar, —C₀-C₆ alkyl-N(C₁-C₄ alkyl)-Het, —C₀-C₆ alkyl-N(C₁-C₄ alkyl)-C₃-C₇ cycloalkyl, —C₀-C₆ alkyl-Ar, —C₀-C₆ alkyl-Het and —C₀-C₆ alkyl-C₃-C₇ cycloalkyl, where x is 0, 1 or 2, or R¹⁴ and R¹⁵, together with the nitrogen to which they are attached, form a 4-7 membered heterocyclic ring which optionally contains one or more additional heteroatoms selected from N, O, and S, wherein said C₁-C₆ alkyl is optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, —CO₂H, —CO₂(unsubstituted C₁-C₆ alkyl), —CONH₂, —CONH (unsubstituted C₁-C₆ alkyl), —CON (unsubstituted C₁-C₆ alkyl)(unsubstituted C₁-C₆ alkyl), —SO₃H, —SO₂NH₂, —SO₂NH (unsubstituted C₁-C₆ alkyl) and —SO₂N (unsubstituted C₁-C₆ alkyl)(unsubstituted C₁-C₆ alkyl);
R¹⁶ is C₁-C₆ alkyl, —C₀-C₆ alkyl-Ar or —C₀-C₆ alkyl-Het; and
R¹⁷ is H, C₁-C₆ alkyl, —C₀-C₆ alkyl-Ar or —C₀-C₆ alkyl-Het;
or a pharmaceutically acceptable salt or solvate thereof.

12. The method according to claim 1, wherein the LXR modulator comprises a compound of formula (IV): 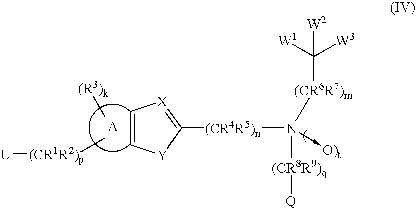
wherein:
X is CH or N;
Y is N(R¹⁰), O, or S, wherein t is 0 or 1 when Y is N(R¹⁰) or O, and t is 0 when Y is S;
U is selected from halo, —OR¹⁰, —NR¹⁴R¹⁵, nitro, cyano, —COOR¹⁰, —COR¹³, —OCOR¹³, —CONR¹⁴R¹⁵, —N(R¹⁴)COR¹³, —SO₃H, —SO₂NR¹⁴R¹⁵, —C(═NR¹⁷)NR¹⁴R¹⁵, —N(R¹⁴)SO₂R¹⁶, and a 5 or 6-membered heterocyclic group;
A is a phenyl fused ring moiety or a pyridyl fused ring moiety, wherein when A is a phenyl ring moiety, k is 0-3 and t is 0 or 1 and when A is a pyridyl ring moiety, k is 0-2 and t is 0;
W¹ is selected from C₃-C₈ cycloalkyl, aryl and Het, wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl, is optionally unsubstituted or substituted by one or more halo substituents;
W² is selected from H, halo, C₁-C₆ alkyl, C₂-C₆ alkenyl, C₂-C₆ alkynyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹²—C₀-C₆ alkyl-NR¹¹COR¹³, —C₀-C₆ alkyl-Het, —C₀-C₆ alkyl-Ar and —C₀-C₆ alkyl-C₃-C₇ cycloalkyl, wherein said C₁-C₆ alkyl is optionally unsubstituted or substituted by one or more halo substituents, and wherein the C₃-C₇ cycloalkyl, Ar and Het moieties of said —C₀-C₆ alkyl-Het, —C₀-C₆ alkyl-Ar and —C₀-C₆ alkyl-C₃-C₇ cycloalkyl are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —CO—C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹², where said C₁-C₆ alkyl, is optionally unsubstituted or substituted by one or more halo substituents;
W³ is selected from the group consisting of: H, halo, C₁-C₆ alkyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹²R¹², —C₀-C₆ alkyl-NR¹¹COR¹³, —C₀-C₆ alkyl-Het, —C₁-C₆ alkyl-Ar and —C₁-C₆ alkyl-C₃-C₇ cycloalkyl, wherein said C₁-C₆ alkyl is optionally unsubstituted or substituted by one or more halo substituents;
Q is selected from C₃-C₈ cycloalkyl, Ar and Het; wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl is optionally unsubstituted or substituted by one or more halo substituents;
p is 0-8;
n is 2-8;
m is 0 or 1;
q is 0 or 1;
t is 0 or 1;
each R¹ and R² are independently selected from H, halo, C₁-C₆ alkyl, C₃-C₆ alkenyl, C₃-C₆ alkynyl, —C₀-C₆ alkyl-NR¹¹R¹², —C₀-C₆ alkyl-OR¹⁰, —C₀-C₆ alkyl-SR¹⁰, —C₁-C₆ alkyl-Het, —C₁-C₆ alkyl-Ar and —C₁-C₆ alkyl-C₃-C₇ cycloalkyl, or R¹ and R² together with the carbon to which they are attached form a 3-5 membered carbocyclic or heterocyclic ring, wherein said heterocyclic ring contains one, or more heteroatoms selected from N, O, and S, where said C₁-C₆ alkyl is optionally unsubstituted or substituted by one or more halo substituents;
each R³ is the same or different and is independently selected from halo, cyano, nitro, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, wherein said C₁-C₆ alkyl is optionally unsubstituted or substituted by one or more halo substituents;
each R⁴ and R⁵ is independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R⁶ and R⁷ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R⁸ and R⁹ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R¹⁰ is selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;
each R¹¹ and each R¹² are independently selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl, or R¹¹ and R¹² together with the nitrogen to which they are attached form a 4-7 membered heterocyclic ring which optionally contains one or more additional heteroatoms selected from N, O, and S;
R¹³ is selected from C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;
R¹⁴ and R¹⁵ are each independently selected from H, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-O—Ar, —C₀-C₆ alkyl-O-Het, —C₀-C₆ alkyl-O—C₃-C₇ cycloalkyl, —C₀-C₆ alkyl-S(O), —C₁-C₆ alkyl, —C₀-C₆ alkyl-S(O)ₓ—Ar, —C₀-C₆ alkyl-S(O)ₓ-Het, —C₀-C₆ alkyl-S(O), —C₃-C₇ cycloalkyl, —C₀-C₆ alkyl-NH—Ar, —C₀-C₆ alkyl-NH-Het, —C₀-C₆ alkyl-NH—C₃-C₇ cycloalkyl, —C₀-C₆ alkyl-N(C₁-C₄ alkyl)-Ar, —C₀-C₆ alkyl-N(C₁-C₄ alkyl)-Het, —C₀-C₆ alkyl-N(C₁-C₄ alkyl)-C₃-C₇ cycloalkyl, —C₀-C₆ alkyl-Ar, —C₀-C₆ alkyl-Het and —C₀-C₆ alkyl-C₃-C₇ cycloalkyl, where x is 0, 1 or 2, or R¹⁴ and R¹⁵, together with the nitrogen to which they are attached, form a 4-7 membered heterocyclic ring which optionally contains one or more additional heteroatoms selected from N, O, and S, wherein said C₁-C₆ alkyl is optionally substituted by one or more of the substituents independently selected from the group halo, —OH, —SH, —NH₂, —NH (unsubstituted C₁-C₆ alkyl), —N(unsubstituted C₁-C₆ alkyl)(unsubstituted C₁-C₆ alkyl), unsubstituted —OC₁-C₆ alkyl, —CO₂H, —CO₂(unsubstituted C₁-C₆ alkyl), —CONH₂, —CONH (unsubstituted C₁-C₆ alkyl), —CON (unsubstituted C₁-C₆ alkyl)(unsubstituted C₁-C₆ alkyl), —SO₃H, —SO₂NH₂, —SO₂NH (unsubstituted C₁-C₆ alkyl) and —SO₂N (unsubstituted C₁-C₆ alkyl)(unsubstituted C₁-C₆ alkyl);
R¹⁶ is C₁-C₆ alkyl, —C₀-C₆ alkyl-Ar or —C₀-C₆ alkyl-Het; and
R¹⁷ is H, C₁-C₆ alkyl, —C₀-C₆ alkyl-Ar or —C₀-C₆ alkyl-Het;
or a pharmaceutically acceptable salt or solvate thereof.

13. The method according to claim 1, wherein the LXR modulator comprises a compound of formula (V): 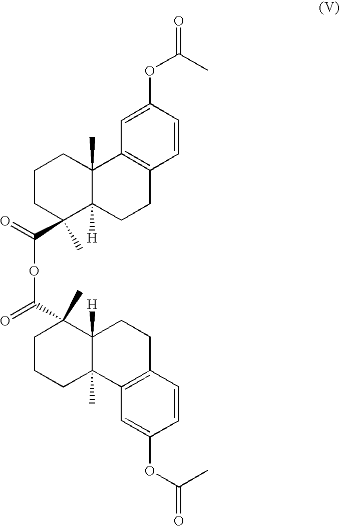
or a pharmaceutically acceptable derivative thereof.

14. The method according to claim 1, wherein the LXR modulator comprises a compound of formula (VI): 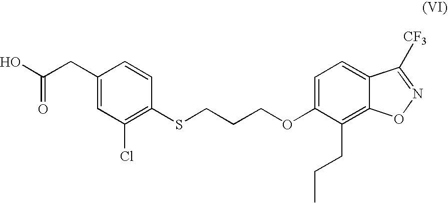
or a pharmaceutically acceptable derivative thereof.

15. The method according to claim 1, wherein the LXR modulator is an LXR agonist.

16-27. (canceled)
